(19) United States
(12) Patent Application Publication
(10) Pub. No.: US 2024/0342098 A1
(43) Pub. Date: Oct. 17, 2024

(54) INGESTIBLE DEVICES, SYSTEMS, AND METHODS THEREOF FOR GASTROINTESTINAL APPLICATIONS

(71) Applicant: UNIVERSITY OF MARYLAND, COLLEGE PARK, College Park, MD (US)

(72) Inventors: Joshua Levy, Lanham, MD (US); Michael Straker, Severn, MD (US); Luke A. Beardslee, Atlanta, GA (US); Reza Ghodssi, Potomac, MD (US); Justin Stine, La Plata, MD (US)

(21) Appl. No.: 18/633,412

(22) Filed: Apr. 11, 2024

Related U.S. Application Data
(60) Provisional application No. 63/495,550, filed on Apr. 11, 2023.
(60) Provisional application No. 63/507,930, filed on Jun. 13, 2023.

(51) Int. Cl. A61K 9/48; A61K 9/00

(52) U.S. Cl. CPC A61K 9/4808; A61K 9/0009; A61K 9/4891

(57) ABSTRACT

Ingestible capsule devices and methods of making these devices are presented. The device and method provide a capsule with embedded actuator for triggerable delivery of drug loaded structures, a freestanding region responsive bilayer (FRRB) comprising a rigid polyethylene glycol (PEG) layer under a flexible pH-responsive layer of methacrylic acid copolymers which protects capsule content until arrival in their target environment of the intestines. The actuator may be fabricated with a flexible cantilever to store mechanical energy and deploy when released using a heater and meltable polymer. The FRRB may be fabricated to form a multitude of shapes that provide functional packaging mechanisms for an underlying ingestible capsule with one or more features, such as openings or seams.

### CROSS REFERENCE TO RELATED APPLICATIONS

The present application is based on, claims priority to, and incorporates herein by reference in their entirety for all purposes, U.S. Provisional Application Ser. No. 63/495,550, filed Apr. 11, 2023, and U.S. Provisional Application Ser. No. 63/507,930, filed Jun. 13, 2023.

### STATEMENT OF GOVERNMENT SUPPORT

This invention was made with government support under ECCS1939236 awarded by the National Science Foundation. The government has certain rights in the invention.

### BACKGROUND

Ingestible devices have the potential to become an attractive tool for use in to overcoming the limitations of traditional means of diagnosis and intervention of many health conditions, including inflammatory bowel diseases (IBD) which affects 6.8 million people worldwide. Currently available ingestible capsules such as the Pillcam™, SmartPill™, and IntelliCap® are capable of general regional drug release, pH, temperature, pressure, and optical sensing in regions of the gastrointestinal tract (GIT) that endoscopes are not, such as the small intestine. However, these products are all generally passive, in that they do not allow external control or interaction with a healthcare provider and/or they do not intelligently react with their environment in a way that allows for precise clinical functions.

Thus, it would be desirable to have a next generation of ingestible devices that can be imbued with functional components that interface with the target environment to perform complex diagnostic and therapeutic functions. Localized drug delivery is an attractive opportunity that could be enabled by ingestible devices. However, no ingestible devices are capable of on-command actuation of movable arms or other members, such as drug-loaded members to release drugs or anchors to allow for localized monitoring or sensing. Moreover, to perform these tasks, many components of ingestible devices would need to be protected from gastrointestinal (GI) fluids or content until they arrive at the intended site of action—then rapidly exposed to the intended site of action.

One attempt at dealing with this issue of targeting a site of action is the use of pH-responsive surface coatings to passively mediate region-specific targeting. Commercially available pH-responsive polymer coatings have been utilized in the packaging of ingestible capsules to facilitate “delayed-release” of drug delivery, sensing, and sample collection components to somewhere further into a GI environment than the esophagus or stomach. These coatings can be used in the pharmaceutical industry for the delayed release of drugs or a payload into general GI regions. For example, thick coatings might not fully dissolve until later in a GI tract when compared to thin coatings or highly pH-responsive coatings. But, this does not provide “on command” deployment of the ingestible's payload. For example, in academic literature, ingestible devices have been postulated as a potential method for gross-localization of monitoring and drug delivery to the GI tract. However, these systems would be triggered merely by passive dissolution and lack the ability to deliver payloads or cause actuation of mechanical members “on command.”

Furthermore, even when ingestibles with dissolvable coatings are used in situations where simple generalized localization is sufficient, merely releasing fluid directly into the GI lumen would still suffer from additional disadvantages that prevent precise deployment of a drug. For example, fluids released directly into the GI lumen are subject to turbulent action in the GI tract hindering localization and preventing full absorption in any given area.

Therefore, it would be desirable to allow for the use of a triggerable actuator to deliver drug-loaded structures like microneedles from an ingestible capsule, and/or to cause on-command anchoring of the capsule, as a possible solution to achieve high levels of drug localization or sensing. Because this approach may involve drug-loaded or water-soluble materials to hold the drugs, protection of the actuator and drug delivery system before deployment becomes an additional design consideration to prevent off-site drug release or leakage. Additionally, in a related sense, protection from luminal content is another design consideration that it would be desirable to achieve, as this can help ensure that the actuation mechanism is not disrupted prior to or during triggering.

Additionally, pH-responsive surface coatings could be adapted for use with multiscale therapeutic carriers for pH specific targeting. This methodology leverages the expansion and contraction of pH-sensitive materials when ionized in response to changes in ambient pH. Enteric coatings utilize polyacids, polymers with functional groups that ionize above a pH threshold, to enable regional targeting. For instance, in the acidic gastric environment (pH 1.5-3) the coatings remain in a contracted state, preventing fluid transport across the coating. Upon arrival in the small intestine (pH 6.4-7.5) or large intestine (pH 6.4-7), the polymer expands resulting in delivery of the payload via dissolution or diffusion mediated mechanisms. Enteric coatings have been applied to ingestible capsules to passively interface with the GI environment for drug or payload delivery and sampling applications, including dissolution for passive region-specific actuator release and collection of ambient media for sample analysis. However, these approaches do not lend themselves to “on-command” actively triggered or site-specific systems at a scale that would be suitable for an ingestible, which would generally use supporting materials to maintain structure or microscale mesh-like openings to avoid component damage during the standard coating process.

Commercially available enteric coatings (such as Eudragit® L100 and similar substances) could be utilized for ingestible capsule devices for use in passive biomarkers sensing and sample collection, utilizing coating methods such as dip coating, spray coating, and pan coating. While these methods are frequently used in pharmaceutical applications, they may not be suitable for electronic capsule coating. Additionally, to dip coat capsules containing functional component ports on the mm-scale, ultra-high viscosity coating solutions would be preferred in many circumstances, resulting in excessively thick coating layers, and impractically slow removal and release times when compared to intestinal transit. Currently, the only alternative to enteric coatings for selectively exposing capsule components, such as sensors and actuators is the addition of an active mechanical opening mechanism. Such mechanisms may require external stimulus from high powered external equipment or cost precious capsule space and energy limited due to size and power source capacity.

### SUMMARY

The present disclosure provides for devices and methods that overcome the aforementioned drawbacks through unique aspects of the structure, function and materials of an ingestible product that enters a person's GI tract. The approaches described herein permit a more precise and/or “on command” deployment of a payload within a specific area of interest within the GI tract.

In accordance with one aspect of the present disclosure, a device is presented. The device comprises a capsule configured to be ingested by a patient and traverse the patient's gastrointestinal tract. The capsule comprises at least two layers forming an outer surface of the capsule: a first water-soluble layer at least partially surrounding the capsule (which covers one or more features of the capsule); and a second pH-responsive layer surrounding the capsule and first water-soluble layer. The capsule also comprises a payload contained by at least a portion of the outer surface of the capsule, the payload comprising at least one electrical or mechanical element.

In another aspect of the present disclosure, a method of making an ingestible capsule is provided. The method comprises forming a film of a first layer that is water-soluble; placing a capsule including one or more active structural features on the film, at least partially wrapping the capsule with the film such that the film covers the one or more features and coating the wrapped capsule in a pH-sensitive layer, the layer comprising one or more coatings.

In another aspect, the present disclosure can include a device including a capsule configured to be ingested by a patient and traverse a patient's gastrointestinal tract. The capsule can include a battery, a switch electrically connected in series with the battery, and a resistive microheater electrically connected in series with the switch. An actuatable arm can have a first end and a second end. The first end of the arm can be affixed to the capsule. An array of microneedles can be coupled to the second end of the arm and can be configured to penetrate intestinal tissue of the patient's gastrointestinal tract upon actuation of the arm. The second end of the actuatable arm can be releasably coupled to the resistive microheater via an adhesive for actuation.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1A illustrates phases of an ingestible capsule from ingestion through deployment and passing, according to aspects of the present disclosure.

FIG. 1B illustrates a magnetic deployment principle of a cantilever system in the presence of a magnetic field, according to aspects of the present disclosure.

FIG. 1C is an example schematic of the cantilever system before, during, and after deployment of a drug, according to aspects of the present disclosure.

FIG. 1D is an example embodiment of a packaged ingestible capsule, according to aspects of the present disclosure.

FIG. 1E is an example embodiment of a dissolving microneedle array releases a drug, according to aspects of the present disclosure.

FIG. 2A is an example schematic of an electrical and packaging system of the ingestible capsule, according to aspects of the present disclosure.

FIG. 2B is an example magnetic reed switch in an open and closed state, according to aspects of the present disclosure.

FIG. 2C is an example assembled ingestible capsule rendering showing a resistive heating elements and overhang used to restrain the fixed end of a cantilever, according to aspects of the present disclosure.

FIG. 2D is an example embodiment of the cantilever, according to aspects of the present disclosure.

FIG. 2E is an example embodiment of the cantilever under the overhang that restrains during flexure and releases when relaxed, according to aspects of the present disclosure.

FIG. 2F is a magnified view of two cantilevers before deployment, according to aspects of the present disclosure.

FIG. 2G is a graph illustrating an example characterization of the magnetic triggering mechanism of the switching field strength and switch resistance for different designed switching strengths, according to aspects of the present disclosure.

FIG. 3A is a graph illustrating an example force profile throughout deployment with representative renderings of the capsule before and after deployment into a load cell, according to aspects of the present disclosure.

FIG. 3B is an example image of the cantilever module after deployment of one cantilever, according to aspects of the present disclosure.

FIG. 3C is a graph illustrating a comparison of the response of PEEK material and UHMWPE material, according to aspects of the present disclosure.

FIG. 3D is a graph illustrating an example deployment profile of different PEEK thicknesses, according to aspects of the present disclosure.

FIG. 3E is a graph illustrating a comparison of PEEK, AC, PES, and PC of 127 μm thickness, according to aspects of the present disclosure.

FIG. 3F is a graph illustrating the relationship between the PEEK cantilever thickness and the actuation force between 76 μm and 508 μm, according to aspects of the present disclosure.

FIG. 3G illustrates several images of the residual angle of cantilevers after prolonged bending, according to aspects of the present disclosure.

FIG. 3H is an example bending apparatus, according to aspects of the present disclosure.

FIG. 3I is a graph illustrating a comparison of relaxation angles between materials of cantilevers have a 127 μm thickness, according to aspects of the present disclosure.

FIG. 3J is a graph illustrating a comparison of relaxation angles between different thicknesses of PEEK cantilevers between 76 μm and 508 μm, according to aspects of the present disclosure.

FIG. 4A is an example illustration of a transition of EVA from solid semi-crystalline translucent state to transparent melt state when heather by a heater, according to aspects of the present disclosure.

FIG. 4B is an example heater packaged in an actuator module, according to aspects of the present disclosure.

FIG. 4C is a 3D map illustrating an areal power density of the heaters as a function of heater diameters and deposition thickness, according to aspects of the present disclosure.

FIG. 4D illustrates six heater photomask designs and fabricated coils, according to aspects of the present disclosure.

FIG. 4E is a plot of the heater resistance of a 70 nm Au trace thickness for heaters of all sizes, according to aspects of the present disclosure.

FIG. 4F is a graph illustrating a comparison of melt times for 2.5 mm heaters with deposition thicknesses of 80 nm, 100 nm, and 140 nm, according to aspects of the present disclosure.

FIG. 5A is an illustration of the deployment of a cantilever actuate while submerged in a solution, according to aspects of the present disclosure.

FIG. 5B is an illustration of the deployment of a drug delivery cantilever in ex vivo intestinal tissue, according to aspects of the present disclosure.

FIG. 5C is an illustration of an evaluation of deployment in a simulated ex vivo environment with controlled translation speed and interfacial forces, according to aspects of the present disclosure.

FIG. 5D is an illustration of the microneedle array 5 minutes after deployment into the intestinal tissue and 2 hours after deployment, according to aspects of the present disclosure.

FIG. 5E is a box and whisker plot illustrating an example cantilever deployment time, according to aspects of the present disclosure.

FIG. 6 is an example fabrication process of the cantilever actuator and integration module, according to aspects of the present disclosure.

FIG. 7A is a schematic of an ingestible capsule from a front view (left) and an isometric view of a portion of the ingestible capsule (right), according to aspects of the present disclosure.

FIG. 7B is a schematic of the ingestible capsule of FIG. 1A including a first water-soluble layer from a front view (left) and an isometric view of a portion of the ingestible capsule (right), according to aspects of the present disclosure.

FIG. 7C is a schematic of an ingestible capsule and first water-soluble layer of FIG. 1B including an outer pH-responsive layer from a front view (left) and an isometric view of a portion of the ingestible capsule (right), according to aspects of the present disclosure.

FIG. 8 is a flowchart of the method of making the FRRB ingestible capsule, according to aspects of the present disclosure.

FIG. 9A is a schematic of packaging system wherein the pH-sensitive polymer coating remains intact and impermeable while in the stomach (pH 1.5-3). In the small intestine, the pH rises (pH 6-7.4) causing polymer swelling and removal. The water-soluble PEG later is then dissolved by the aqueous intestinal environment, thus revealing the capsule actuator cavity.

FIG. 9B is a schematic of the chemical structure of Eudragit® L 100 55 at stomach and small intestinal pH. In the stomach the structure is protonated, and polymer is compact while in small intestinal conditions, the structure is deprotonated causing monomers to repel each other and expand.

FIG. 10 is a workflow of the bilayer coating process: 1) A polyimide sheet is placed on a hotplate and polyethylene glycol (PEG) crystals are melted into a liquid film. 2) 3D printed polyethylene terephthalate glycol (PETG) capsule is presented onto PEG film. 3) The polyimide and PEG layers are folded over the capsule and cooled until PEG film solidifies. 4) The polyimide film layer is readily peeled off. 5) Sample is dip-coated in pH-responsive polymer.

FIG. 11A is a CAD design of select function capsule geometries possible with the FRRB, specifically, the fabrication (left) and removal (right) of capsule including a slot coated with the FRRB. The actuator slot design is used to demonstrate the release of capsule actuator for GI wall access.

FIG. 11B is a CAD design of select function capsule geometries possible with the FRRB, specifically, the fabrication (left) and removal (right) of capsule with a dome-shaped sheath coated with the FRRB. The dome shaped sheath can be molded to cap the end of a capsule leaving room for large area opening.

FIG. 11C is a CAD design of select function capsule geometries possible with the FRRB, specifically, the fabrication (left) and removal (right) of capsule including multiple ports coated with the FRRB. The multiport capsule has three layers of ports which are sealed by the FRRB and exposed at different times.

FIG. 11D is a CAD design of select function capsule geometries possible with the FRRB, specifically, the fabrication (left) and removal (right) of capsule including made of two halves coated with the FRRB. The FRRB is used to connect the two halves of the capsule, which are separated upon removal of the coating.

FIG. 12A is a schematic of a cross section of coated test capsules prior to and after penetration (top) and the corresponding circuit schematics of the test setup (bottom).

FIG. 12B is a plot of an example of the raw impedance data collected during the bath penetration experiments (1× coated sample) showing the sudden drop in impedance, indicating penetration time.

FIG. 12C is a bar graph of acetate penetration of the bilayer with multiple pH dependent coating layers (0, 1, 3, 5, 10). The x-axis represents the number of layers of Eudragit® FL 30 D-55 used in the coating of the sample. Error bars represent standard deviation. The dotted line at 180 min shows the average gastric emptying time expect of normal fed systems and the dotted line at 30 min marks average gastric emptying time of a fasted system.

FIG. 12D is a bar graph of penetration time in neutral pH. The dotted line at 260 min denotes the average small intestinal emptying time. (n=3).

FIG. 13A is a plot of the linear fit of the bilayer penetration time. The dotted line represents the time of exiting the duodenum (pH 7).

FIG. 13B is a plot of the linear fit of the bilayer penetration time. The dotted line at t=30 min represents gastric emptying in a fasted system while the dotted line at t=180 min represents the maximum gastric emptying time (pH 3).

FIG. 14A is a flowchart showing a capsule with a white paper indicator packaged inside coated with FRRB (5×FL30). The capsule is then incubated in an acid bath (pH 3). It is then translated across the tissue phantom in neutral solution (pH 7).

FIG. 14B is a photo of paper strips protected or release by the capsule after four conditions: 1) Blank/no incubation; 2) 30 minutes acid incubation and exposed in neutral solution on the GI simulator. 3) Negative control/acid solution. 4) Both acid and neutral solutions. Scale bars: 5 mm.

FIG. 15A is a plot of the coating thickness of Eudragit® FL 30 D-55 at 1, 10, and 30% w/v.

FIG. 15B is a bar graph of the FRRB removal time in the ex vivo intestinal simulator fabricated with PEG and 1, 10, and 30% w/v Eudragit® formulations. The dotted line at 150 min represents the expected small intestine transit time.

FIG. 16A is a photo of the capsule before ex-vivo intestinal simulator testing from a front view (top) and side view (bottom). Before testing, capsules have uniform surface coatings over actuation cavity.

FIG. 16B is a photo of the capsule after ex-vivo intestinal stimulator testing in a front view (top) and side view (bottom. After testing, images show complete removal of the FRRB. Dashed lines indicate the capsule perimeter.

FIG. 17A is a series of images of capsules before (top) and after (bottom) translation across a tissue phantom (left: dome capsule; middle: connector capsule; right: multislot capsule). Scale bars: 5 mm.

FIG. 17B is a bar graph of removal times for each geometric design including the total time to exposure of each port of the multiport capsule form the start of translation (n=3).

FIG. 18A is a diagram of an example application of the FBBR capsule upon bilayer coating removal. The pH=sensitive polymer coating remains intact and impermeable while in the stomach (pH 1.5-3). In the small intestine, the pH rises (pH 6-7.4) causing polymer swelling and swift removal. The water-soluble PEG layer is then dissolved by the hydrated intestinal environment, thus revealing the capsule actuator cavity.

FIG. 18B is a diagram of a spring actuator and drug delivery structure within the capsule of FIG. 18A.

FIG. 19 is a diagram of the ex-vivo gastrointestinal simulator system. Translation is facilitated by the screw lead motor which moves the rail-mounted platform attached to the capsule holder. A dish holding a 50 g mass is attached to the top of two-pronged mass loaded capsule holder while coated test capsule is fastened to the bottom. This applies a total of ˜70 g of mass from the top of the capsule to the tissue phantom placed in the phantom tissue holder to approximate circumferential peristaltic forces in the GI tract.

### DETAILED DESCRIPTION

The present disclosure devices and methods that overcome the aforementioned drawbacks. For example, some embodiments may overcome limitations of existing approaches by providing an actively triggered actuator that is protected by a passive freestanding hybrid packaging technology. The actuator may comprise a simple cantilever mechanism that unflexes to inject drug-loaded microneedle arrays or other drug structures into intestinal tissue. The system may use a thin film microfabricated heater to melt the adhesive holding the cantilever in place, deploying the actuator. The cantilever may be embedded in a capsule to hold and deploy this cantilever actuator system using a battery and magnetic switch enabling deployment when exposed to a nonspecific magnetic field, such as that produced by a handheld NdFeB magnet held to the body. The system is simple in design and operation, and uses onboard power and switching for deployment, lending potential for multi-system integration and sensor-triggered deployment in the future. Protection of the cantilever actuator is accomplished using the bilayer described below.

Furthermore, a bilayer for targeted exposure of ingestible capsule components can further aid in achieving targeted deployment of such a payload. For example, the bilayers disclosed herein allow for unsupported coverage of mm-scale openings where pH responsive polymer coatings alone cannot. Various devices and methods herein may utilize a flexible pH responsive Eudragit® FL 30 D 55 layer paired with a rigid water-soluble polyethylene glycol (PEG) support layer to form the freestanding region-responsive bilayer (FRRB) which can protect and selectively expose mm-scale openings in ingestible capsules. The PEG support, which may be formed by melt processing, provides a conformable substrate to enable geometries that were previously unattainable using dip coating. The rigid PEG support also allows the FRRB to protect the underlying components without contacting them while facilitating exposure in response to pH-specific regions along the GI tract. The bilayer eliminates the need for complex space-and energy-intensive opening mechanisms. Without the rigid freestanding layer, opening sizes and geometries are limited by the constraints of conventional dip coating or space and energy consideration.

### Cantilever Actuator Module

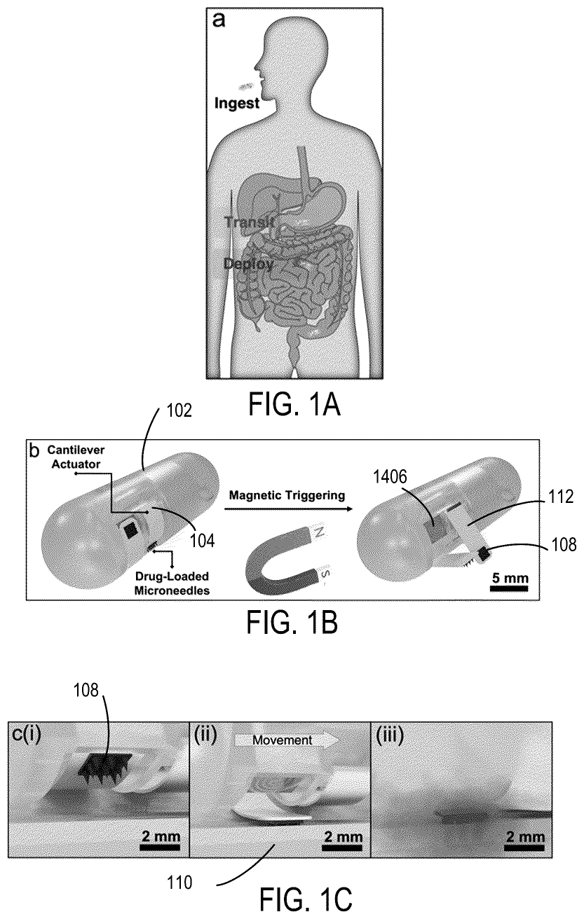

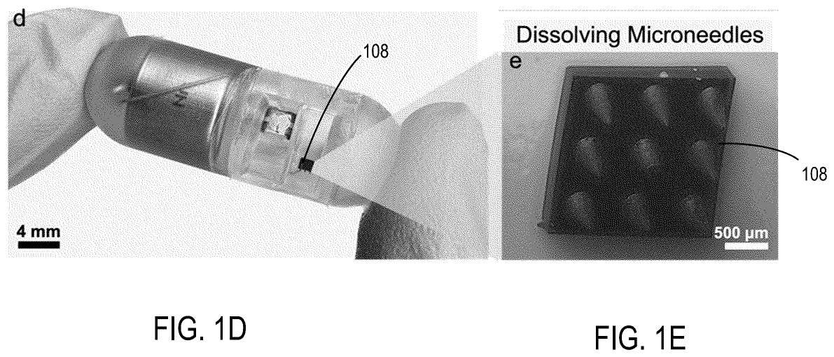

Referring to FIGS. 1A-E, different aspects of a capsule 102 are shown. In some examples, the capsule 102 is configured to be an ingestible capsule. In other examples, the capsule 102 may comprise two or more complementary pieces that are configured to reversibly connect to form the capsule 102.

Further, the capsule 102 can comprise an actuatable arm. As illustrated in FIGS. 1A-E, the actuatable arm is a cantilever actuator 104. In other examples, the actuatable arm can include a piston or dart that move perpendicular to the longitudinal axis of the capsule. The capsule 102 can further comprise a heating element 106, and an array of microneedles 108, In some examples, the array of microneedles 108 may separate from the body of the capsule 102 and penetrate the intestinal tissue 110 of a patient. In some examples, the array of microneedles may be attached to one end of a cantilever 112. The cantilever may be attached to the body of the capsule 102 via the cantilever actuator 104 in combination with an adhesive element. The separation of the microneedles 108 may occur when a magnetic field is triggered, causing the heating element 106 to melt the adhesive element. In some examples, the array of microneedles may dissolve after an amount of time. In some non-limiting examples, the capsule 102 may include one or more of the above-mentioned elements.

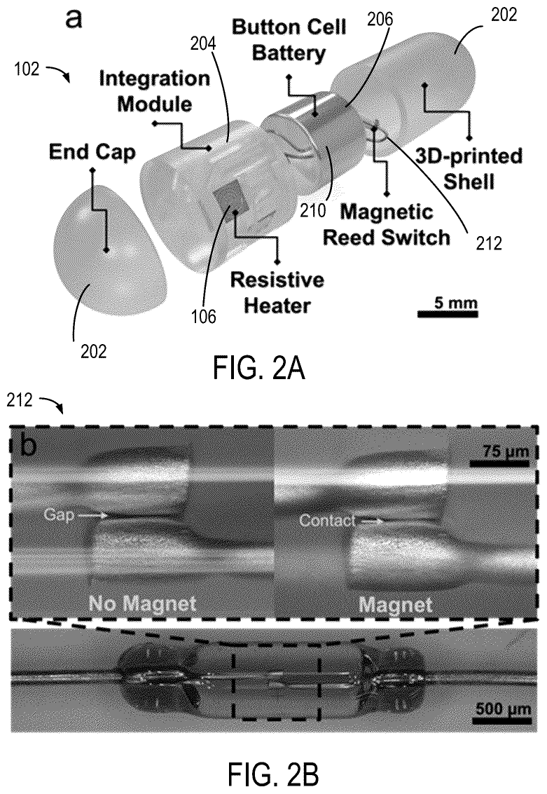

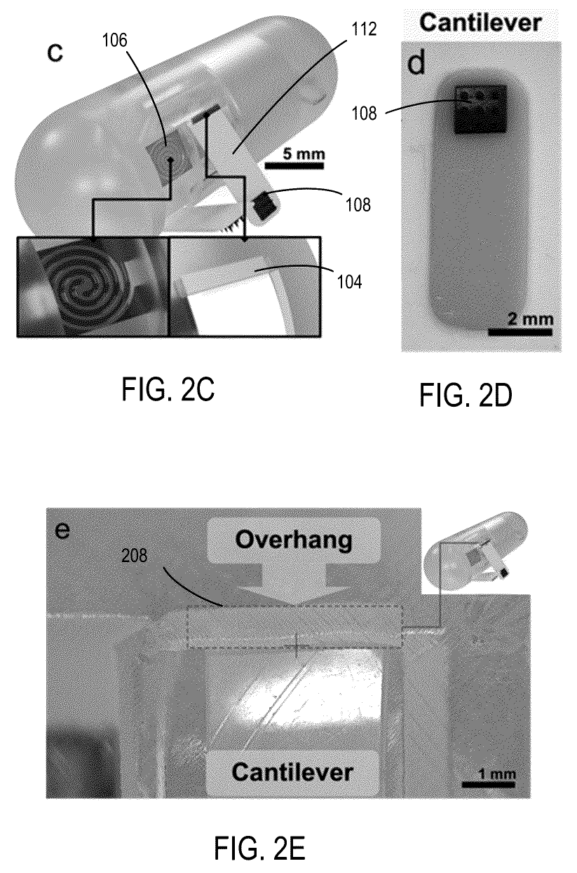

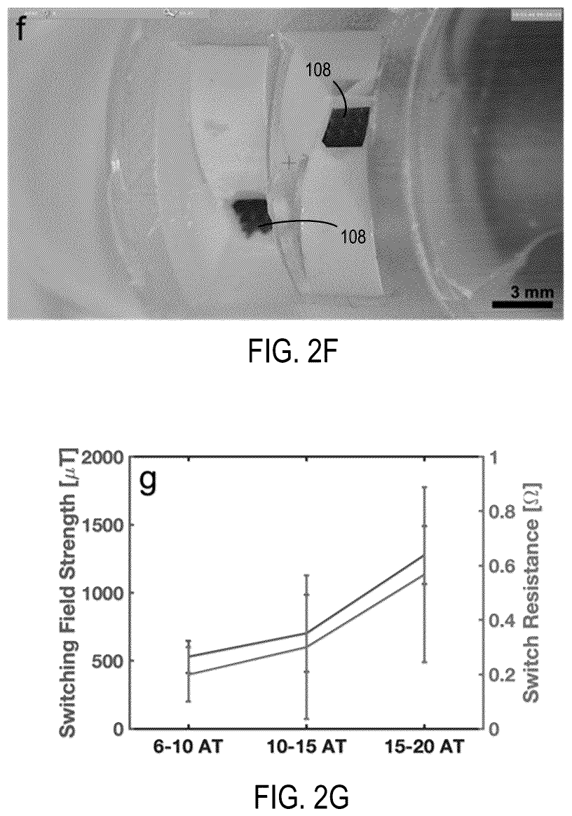

Referring now to FIGS. 2A-G, different aspects relating to modules of the capsule 102 are shown. In some embodiments, the capsule 102 further comprises an end cap 202, an integration module 204, and a power switching module 206.

In some embodiments, the integration module 204 can be configured as a portion of the internal payload of the capsule that contains elements that provide for electromechanical actuation of an arm or other movable member of the capsule. Thus, the integration module 204 may comprise the heating element 106, the cantilever actuator 104, the array of microneedles 108, and the cantilever 112. In some examples, the heating element 106 may be configured as a resistive heater. Moreover, the cantilever 112 may be coupled to the cantilever actuator 104 via a fixation point, such as a tab, adhesive, or an overhang 208. In some examples, the cantilever 112 may detach from the integration module 204 during deployment. In some examples, the overhang 208 may aid in the cantilever 112 detaching. Before the cantilever 112 is deployed, the force associated with the flexion and/or compression of the cantilever 112 may be applied to the overhang 208, keeping the cantilever 112 attached to the integration module 204. However, that force may no longer be applied to the overhang 208 during deployment, allowing the cantilever 112 to fall off of the integration module 204 in embodiments where such detachment may be desirable. In other embodiments, it may be desirable for the cantilever to remain affixed to the capsule, whether to serve as an anchor or to allow for drug release while the whole capsule remains in place.

The power switching module 206 may comprise a battery 210, a magnetic reed switch 212, and a connection to the heating element 106. In some embodiments, the heating element 106 may be configured as a resistive microheater. The battery 210, magnetic reed switch 212, and heating element 106 may be electrically connected in series. When a magnetic field of a specified strength is applied to the power switching module 206, the magnetic reed switch 212 may close, allowing the battery 210 to power the heating element 106. In some examples, the magnetic field is applied externally. Other mechanisms for achieving power switching, aside from a magnetic switch, are also contemplated and described in other examples below.

In accordance with one aspect of the present disclosure, a device comprising a capsule, a power source, a bioimpedance sensor, and a transmitter is described. The capsule is configured to be ingested by a patient and traverse the patient's gastrointestinal tract (GI). In a non-limiting example, the capsule is cylindrical with a diameter between 4-20 mm and length between 1-35 mm. Capsule diameter is linked to contact pressure. For an ingestible capsule, the maximum diameter of the capsule is limited by the esophagus, which is the narrowest segment of the GI tract. Thus, capsule size and profile may be determined so as to allow for comfortable swallowing by an average human. However, in some embodiments, larger sizes may be useful (while still permitting passage through the esophagus) even though swallowing may not be comfortable, as ingestible devices may not be swallowed as frequently as other items, such as dissolvable medication capsules.

In some example implementations, the cantilever actuator capsule system may comprise a magnetic reed switch in series with the 2L76 button cell battery and resistive microheater to trigger actuation when exposed to a magnetic field (FIG. 1B). The heater may be fabricated by a standard liftoff process on a Kapton® substrate. The reed switch may be electrically joined to the battery via spot-welding, and electrical connections between the heater, battery, and reed switch are formed via soldering and silver conductive paste filled channels in the cantilever module. The capsule shell may directly attach to the cantilever module and contains the battery, reed switch, and associated wiring, while the cap attaches to the other side of the module to create a rounded end shape (FIG. 2A). The capsule shell, cap, and cantilever module may all be 3D printed using an LCD vat photo-polymerization (VPP) process of biocompatible resins, and the capsule is assembled by attaching the shell and caps to the cantilever module using biocompatible urethane adhesive. Cantilevers may be attached on the fixed end using loose restraints in the module that hold during cantilever flexure but allow release after deployment. The free end of the flexed cantilever may be held in place by a low melting point adhesive (EVA) that melts when heated by the heater. Attached to the free end of the cantilever is a 3×3 drug-loaded microneedle array that penetrates the intestinal tissue releasing drug following deployment. The cantilever actuator can be triggered by the resistive heater melting the adhesive holding the cantilever in place. Current flow through the heater is controlled by the magnetic reed switch connecting the heater and battery (FIG. 2A).

Magnetic Triggering: The triggering mechanism for an ingestible capsule may rely on the state of the magnetic reed switch to initiate cantilever actuation. FIG. 2B shows the magnetic reed switch composed of a glass package containing inert gas, ferromagnetic reeds, and leads exiting the glass package. The unperturbed open (left) and polarized closed state (right) show the gap and contact, respectively, of the two reeds. When in a magnetic field, the two ferromagnetic reeds suspended in the glass package generate temporary magnetic polarization, attracting each other and forming electrical contact. To evaluate the effectiveness of the reed switch in the given environment, this contact was assessed on the benchtop and the amount of magnetic field to be reliably used for switching and switch resistance were evaluated. The switching field strength and switch resistance were evaluated (FIG. 2G) to understand the impact of the designed switching strength, measured conventionally in Ampere-turns (AT), on these characteristics. Magnetic reed switches with designed switching strength of 6-10 AT, 10-15 AT, and 15-20 AT exhibited ascending switching field strength as expected. 6-10 AT switches closed at 529±117 μT (n=3), 10-15 AT switches closed at 702±282 μT (n=3), and 15-20 AT switches closed at 1278±212 μT (n=3). This range of field strength is suitable because it exceeds commonly encountered magnetic fields but is readily achievable with handheld magnets or portable EM generating devices. For example, Earth's magnetic field strength varies between approximately 20 μT and 68 μT, and commercial medical equipment utilize up to 1.5 T. Moreover, these μT magnetic fields exhibit little magnetic force pulling on the capsule; at a 1 mT field the capsule generated <10 mN of force on the capsule, far less than that required to begin damaging intestinal tissue. The switch resistance also follows a similar trend, ascending with switch strength. The immediate reason for this is unclear, though it may result from the increased reed rigidity or experimental variations. While switch resistances exhibited relatively large standard deviation, the resistance remained below 1Ω, significantly less than the heater (≥50Ω), resulting in a negligible impact on the power dissipation of the heater. While all examined reed switches are suitable and show minimal potential for bodily harm, these results demonstrate that the 6-10 AT switch is ideal due to its lower switching field strength and switch resistance, increasing the likelihood of successful deployment.

In other examples, a switch may be triggered remotely using a printed circuit board (PCB) having a microprocessor that is in communication with a mobile device or other controller in a healthcare facility, such as via Bluetooth or other radio frequency communication protocols. Upon receiving a signal from the controller, the PCB can electrically close a circuit (e.g., via an electrical switch such as a transistor) between the heating element and battery. In other examples, the cantilever may be deployed by dissolving a polymer rather than melting.

Mechanical Analysis of the Cantilever: The cantilever actuator stores elastic potential energy by flexure of the cantilever about the capsule circumference and is designed to deploy by melting of the adhesive EVA followed by detachment from the capsule after deployment. FIG. 2C shows a magnified view of the heater and cantilever in position, where the fixed end of the cantilever can be seen resting under the overhang in the cantilever module, enabling fixture during compression and detachment after deployment. FIG. 2D shows the cantilever with attached drug loaded microneedles, an 8.5×3 mm rectangular shape with 1 mm fillets to eliminate sharp edges, precluding tissue damage after deployment. The cantilever actuator relies on the internal stresses in the flexed cantilever to deploy rapidly with the appropriate force. The success criteria can include (1) achieving rapid actuation (<5 s) to target specific locations before moving significantly due to intestinal contractions and (2) achieving suitable force to insert microneedles without damaging intestinal tissue (100-500 mN) given the 2×2 mm microneedle array patch. To evaluate the response time and force dynamics, mechanical tests were performed by deployment of the cantilever into a load cell.

The following materials are contemplated as useful for forming a device according to the present disclosure, given their biocompatibility and specific bulk mechanical properties (i.e., high modulus and resistance to stress relaxation): Polyether ether ketone (PEEK), cellulose acetate (AC), polyester (PES), polycarbonate (PC), and ultra-high molecular weight polyethylene (UHMWPE). The materials identified by the inventors, in their bulk form, have advantageous mechanical properties. Nevertheless, the performance of these different materials in the context of this mechanism is not entirely predictable.

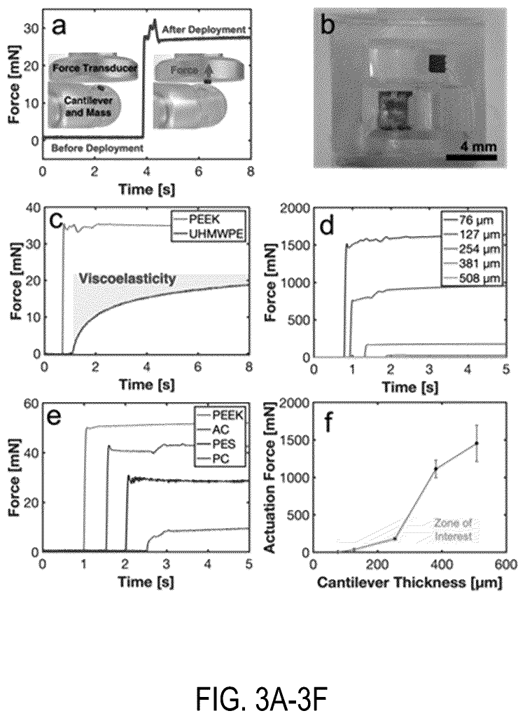

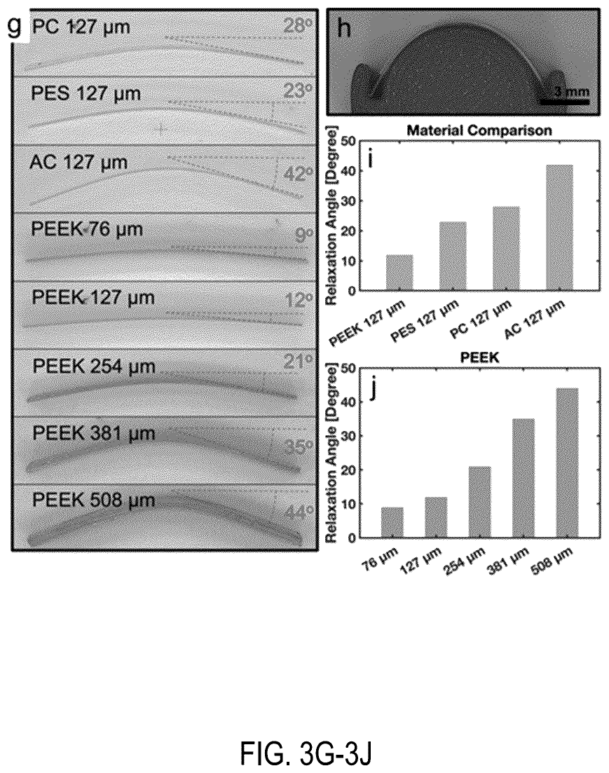

Thicknesses in the range of 75-500 μm, or in the range of 100-400 μm, or in the range of 200-300 μm, and in some embodiments a cantilever made of PEEK material with a thickness of approximately 254 μm were determined to be suitable to achieve the desired forces. In some examples, the thickness may be determined using the modulus of the cantilever material and/or the system to which the capsule is delivered. FIGS. 3A-3F show the results of mechanical tests of various cantilever materials and thicknesses. For all cases, a 10 mg proof mass was used to approximate the microneedle array weight and inertial contributions without influencing the results due to dampening by the microneedle structure. FIG. 3A shows a representation of the deployment experiment before deployment (t<4 s) and after deployment (t>4 s) with the accompanying force profile for a 127 μm PEEK cantilever. In some embodiments, it may be advantageous to have cantilever deployment occur rapidly, but this can result in a peak force higher than the equilibrium force due to the inertia of the mass and cantilever. The force then equilibrates to a steady state within milliseconds resulting from the elastic cantilever force on the load cell. While this is evident in the case of PEEK (FIG. 3A), other materials tested exhibited gradual deployment and progressive force profiles on the load cell (FIG. 3C). This is due to the viscoelastic properties, specifically stress relaxation, of the polymeric cantilevers. FIG. 3C highlights this effect showing the difference between the rapid deployment of a PEEK cantilever and gradual deployment of a UHMWPE cantilever. For elastic applications, high viscoelastic character due to stress-induced molecular rearrangements is an inherent challenge for polymers; this is not only because it results in slow deployment, but also because the extent of relaxation changes with time, making corrections for prolonged deployment time impossible. Overall, thicker cantilevers are more susceptible to stress relaxation because they are subject to higher compressive and tensile strains on the bottom and top surfaces, respectively. However, the thicker cantilevers also exhibit higher forces, as evidenced by FIGS. 3D and 3F comparing the deployment force of varied thicknesses of PEEK between 76 μm and 508 μm (n=3).

The challenge of stress relaxation of polymeric structures is a factor that is overcome by embodiments described herein that utilize a tensioned cantilever, compressed spring, or other member having stored kinetic energy. While polymers interface well with the soft intestinal tissue, they are susceptible to the aforementioned viscoelastic properties. To specifically assess long-term transience of deployment, cantilevers underwent prolonged bending at 130° for 72 hours in a 3D-printed bending apparatus meant to emulate bending in the capsule as shown in FIG. 3H. Following the 72 hours, bending images revealed the residual angle of the cantilevers (FIG. 3G). As expected from its high modulus and resistance to stress relaxation, PEEK outperformed other materials (PC, PES, AC), and thicker cantilevers exhibited further relaxation. Thus, PEEK was the chosen material for this application. The 254 μm PEEK cantilevers presented the ideal balance of deployment force, 180.8±17.0 mN (n=3), and relaxation angle, only 21° over 72 hours of bending. Furthermore, the 21° initial relaxation did not proceed proportionately over a 60-day period, resulting in only 32° of relaxation after the 60 days in flexure. These viscoelastic characteristics make the 254 μm PEEK cantilever an excellent choice for this application, delivering rapid deployment response and prolonged shelf life compared to prior examples of similar technologies.

Resistive Heater Design: The restive heating element is a component that can allow for controllable deployment time of the cantilever actuator by causing melting of the EVA adhesive (or other adhesive) that holds the cantilever in flexion. Design and optimization of the heater must account for input power constraints, deployment time, and reliability. In a prior development of a similar thin-film heating mechanism, the heater resistance was controlled by modifying the deposition thickness of the Au traces. To achieve the fastest deployment time with the given geometry, the resistance was tuned to ˜50Ω based on the peak discharge current (60 mA) achievable from the 2L76 coin cell battery and assuming a ˜3V potential difference. This configuration resulted in the highest achievable power dissipation to achieve rapid melting of the polymer adhesive. Nevertheless, reduction of heater size and tuning of current supply could enable higher areal density of thermal dissipation leading to faster melting, lower power consumption, and greater potential for future device miniaturization. To this end, further evaluation of case examples of heater geometry and power constraints was performed to optimize melt time, efficiency, and reliability.

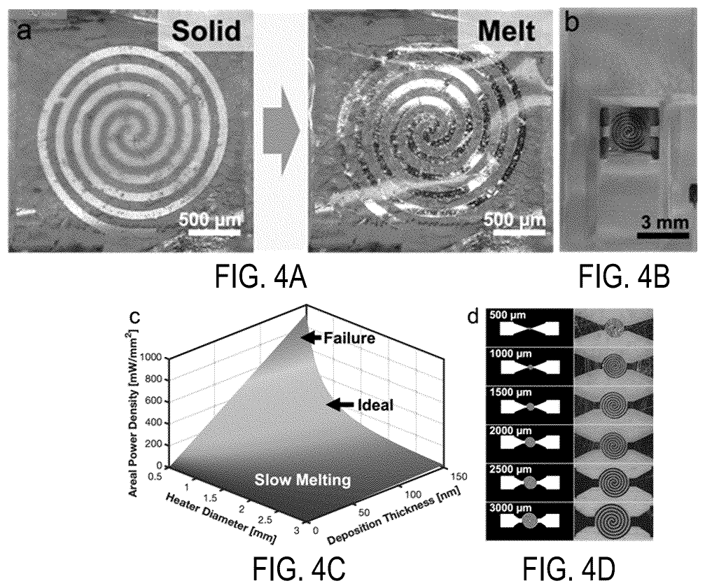

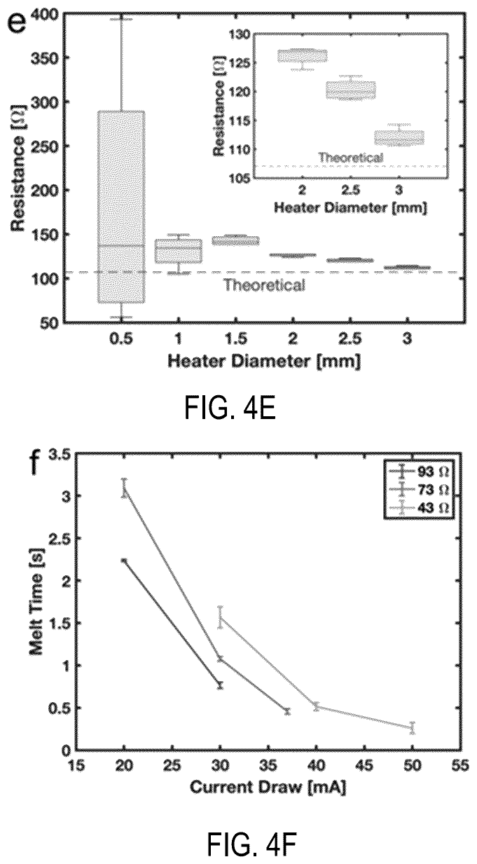

Heaters were designed in six different variants, all having the same proportionate geometry, with different sizes shown in FIG. 4D (outer diameter (OD) of: 0.5 mm, 1 mm, 1.5 mm, 2 mm, 2.5 mm, and 3 mm). An example of the heater with 2.5 mm OD is shown in FIG. 4a before and after melting the EVA adhesive, and in FIG. 4B after integration with the cantilever module of the capsule by electrical connection using Conductive Ag Epoxy. Due to the identical heater geometry across sizes, and thus ratio of trace length (L) to trace width (W), the resistance was expected to be the same for each variant with the same Au deposition thickness. Evaluation of the heater was performed via modelling and benchtop testing. FIG. 4C shows a plot of the theoretical areal power density in mW/mm². At comparable resistance, smaller heaters dissipate the same amount of energy over a smaller area leading to higher areal power density and higher heater surface equilibrium temperatures. Additionally, the deposition thickness linearly impacts the cross-sectional area of the heater and thus inversely impacts the resistance. Thicker traces with lower resistance result in higher current flow and power. While higher areal power density does not linearly correlate to efficiency, smaller heaters reduce wasted thermal energy when used with smaller adhesive quantities and are expected to minimize melt time, further reducing wasted thermal energy. The areal power density also has implications on reliability, where higher temperatures seen in smaller heaters resulted in frequent electrothermal trace failures. The highest areal power density achievable with the evaluated sizes and power constraints is a ˜900 mW/mm² with a 0.5 mm heater and 150 nm trace thickness. Heaters with less than ˜200 mW/mm² proved to be most reliable.

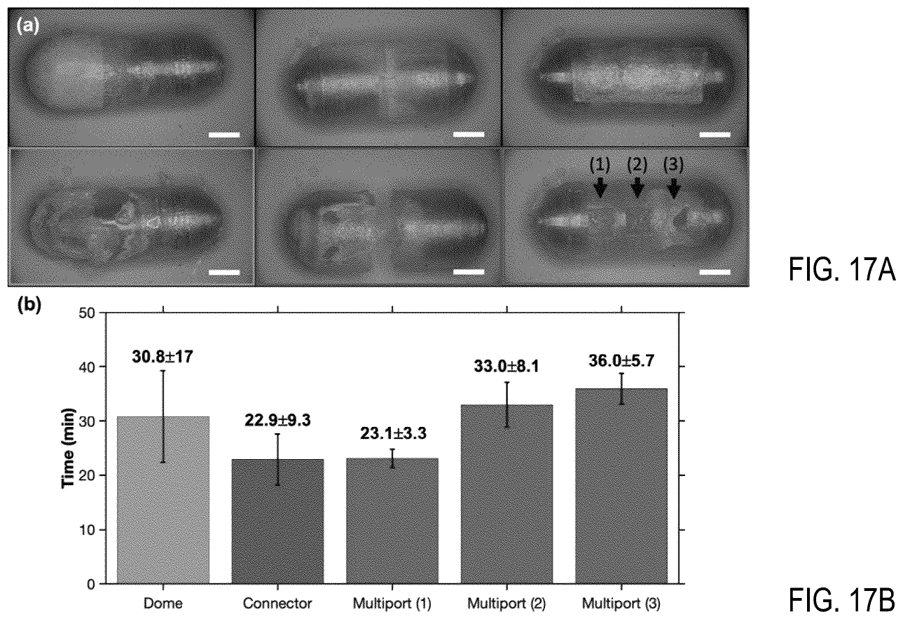

To identify general trends in the reliability and deployment time, the heaters were evaluated on the benchtop. FIG. 4E shows the characterization of heater resistance for all sizes of a 70 nm Au trace thickness heater to illustrate trends seen across all groups. Overall, the smaller heaters were less reliable and less predictable. As can be seen in FIG. 4E, the 0.5 mm heater had a mean resistance of 180.8±151.9Ω (n=4), while the 3 mm heater had a mean resistance of 112.0±1.6Ω (n=4). Moreover, the larger heaters performed closer to the expected theoretical performance, with the 3 mm heater deviating only 4.6% from the theoretical value of 107.1Ω predicted in FIG. 17C. Thus, while the smaller heaters achieve a desirably high areal power dissipation density, the lower limit of heater size should be ˜1 mm OD to minimize failure and unpredictability. To assess the impact of heater resistance on melt times, various thicknesses of 2.5 mm heaters were evaluated under controlled current using a commercial power supply regulated to 3.3 V (n=3). As can be seen in FIG. 4F, higher current draw generally resulted in faster melting; however, heaters of higher resistance performed better at a given current limitation. These results are unsurprising, but they validate that heaters of higher resistance enable balancing of energy consumption and deployment time. These results indicate that ideal heater configuration to achieve deployment in less than 1 s with minimized power consumption is a size between 1 mm and 2.5 mm, and a trace thickness of ≤80 nm for embodiments using a low-melt adhesive and tensioned cantilever. Deployment in 3 sec supports precise sub-mm delivery location targeting in the intestinal tract considering a mean intestinal translation speed of 1.4 cm/min. It should be understood, however, that ideal heater configuration is a design consideration impacted by the specific adhesive that is to be melted, the tension or force stored in the cantilever (or spring, or other member), and their structural tolerance. Thus, in line with the example discussed herein, other “ideal” heater configurations are contemplated for other adhesives, batteries, and armatures as well.

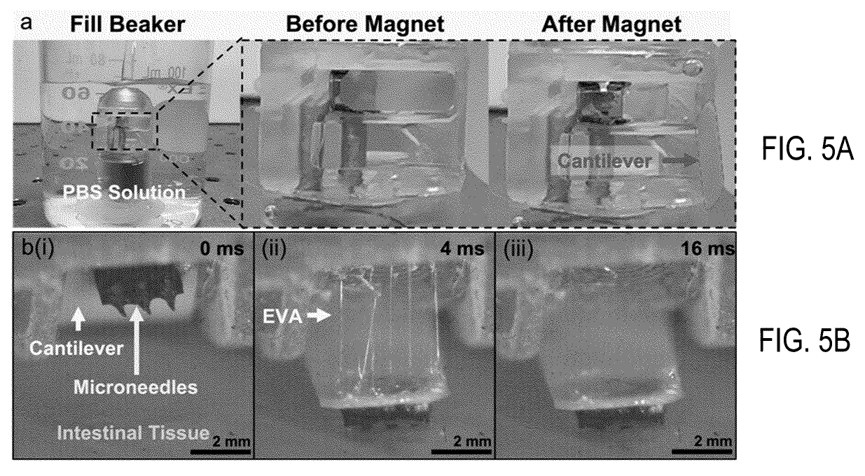

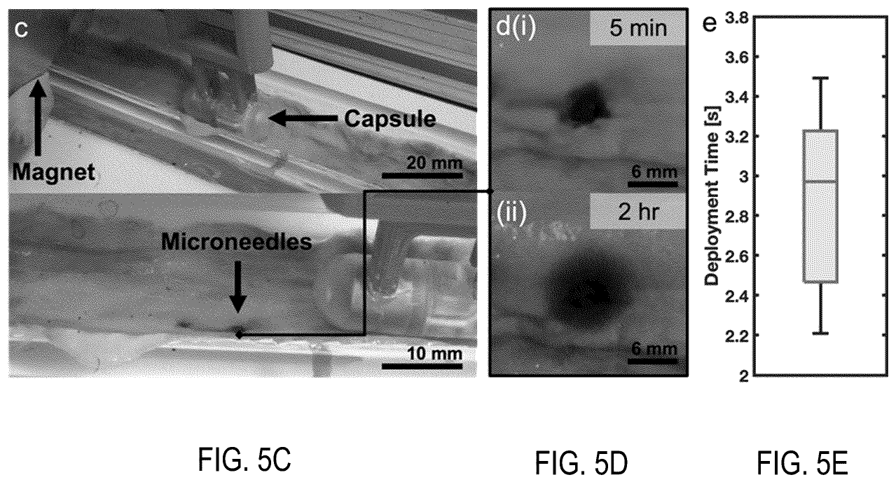

Benchtop and Ex Vivo Validation of Drug Delivery: To assess the performance of the localized drug delivery capsule in a realistic environment, the actuator was deployed while submerged in 1× phosphate buffered saline (PBS) solution and in a porcine ex vivo intestinal environment. FIG. 5A shows the capsule undergoing testing while submerged to verify its resilience to operation in an aqueous environment like the gut. The capsule was placed in a beaker and submerged, then a handheld NdFeB magnet was used to trigger the actuation of the cantilever. No solution was observed to permeate the capsule shell over the course of 1 hour, and the cantilever actuator deployed in <3 s after introduction of the magnet within 10 cm of the capsule. Full submersion is intended to simulate an extreme case of full submersion in the intestine, as the capsule will likely encounter a combination of liquid and gaseous media in transit through the esophagus, stomach, and small intestine. While this demonstration confirmed that actuator deployment is possible while fully submerged, protection of the drug loaded microneedle array was not demonstrated. Previously, our research group has demonstrated a freestanding bilayer for protection of active components for ingestible device. In the future, we will use this bilayer to protect the microneedles from premature drug release.

Deployment of the capsule in ex vivo intestinal tissue is intended to simulate the close to realistic conditions expected at the interface between the cantilever, microneedles, and gut environment. The microneedle penetration into intestinal tissue was characterized using a high-speed camera to better understand the interaction of the actuated cantilever and microneedles with intestinal tissue during deployment (FIG. 5B). FIG. 5B show frames from the 1000 fps video of deployment of the actuator and microneedles into the ex vivo porcine small intestinal tissue after the EVA adhesive begins to melt. In FIG. 5B(i) (t=0 ms), the cantilever with attached microneedles is in the flexed state attached to the heater. After 4 ms (FIG. 5B(ii)), the cantilever has almost fully deployed showing stringing of the EVA adhesive that previously held the cantilever in flexure. After a total of 16 ms, the cantilever is fully deployed, and the drug loaded microneedles (FIG. 5B(iii)) have penetrated the surface of the tissue. Thus, considering individual characterization the total deployment time was estimated as a combination of the 500-1000 ms heater melt time and the 16 ms cantilever motion. However, in experimentation with magnetic control, the capsule exhibited deployment in 2.91±0.48 s (n=7) (FIG. 5E). This deviation from the expected deployment time is believed to be due to a combination of switch and system inefficiencies, like internal resistance and thermal dissipation to the surrounding aqueous media. Nevertheless, an 85% reduction in deployment time was exhibited compared to the prior actuation prototype demonstrated in literature, enabling significantly greater location targeting and longevity.

Following the close-up analysis of deployment in a static ex vivo environment, the localized drug delivery capsule was evaluated on a custom gastrointestinal simulator described in Straker et al. The simulator is composed of a compliant polydimethylsiloxane (PDMS) tissue bed molded to a curvature of r=12.5 mm, and a screw-rod translation system for capsule mounting. Tissue is adhered to the surface of the PDMS bed during testing, thus adopting the approximate curvature of the small intestine. The speed of transit was controlled to 1.4 cm/min, the mean translation speed in the small intestine. The capsule mount has vertical freedom, allowing mass loading to control the force on the capsule and approximate peristaltic and segmentation contractions of the gut. During translation, a magnet was used to trigger deployment of the cantilever, leaving the dye-loaded microneedle array behind (FIG. 5C). Following detachment of the microneedles via rapidly dissolving PEG polymer, the release of dye was monitored over the course of 2 hours. FIG. 5D shows the microneedle array 5 minutes after deployment, and after 2 hours the dye spread was observed at a diameter of ˜10 mm from the 2×2 mm needle array (FIG. 5D(ii)). Overall, this level of confinement enables significantly higher drug concentrations with minimized dosing. For example, with 1% of the drug loading of a small intestine targeted oral tablet, this delivery system can achieve ˜380× areal drug concentration at sites of need compared to broad distribution across the surface area of the intestinal tract. While uncontrollable factors like diffusion and turbulence in the gut will attenuate the level of localization demonstrated here, this illustrates the potency of such an approach to improve local treatment while significantly mitigating systemic drug exposure and side effects.

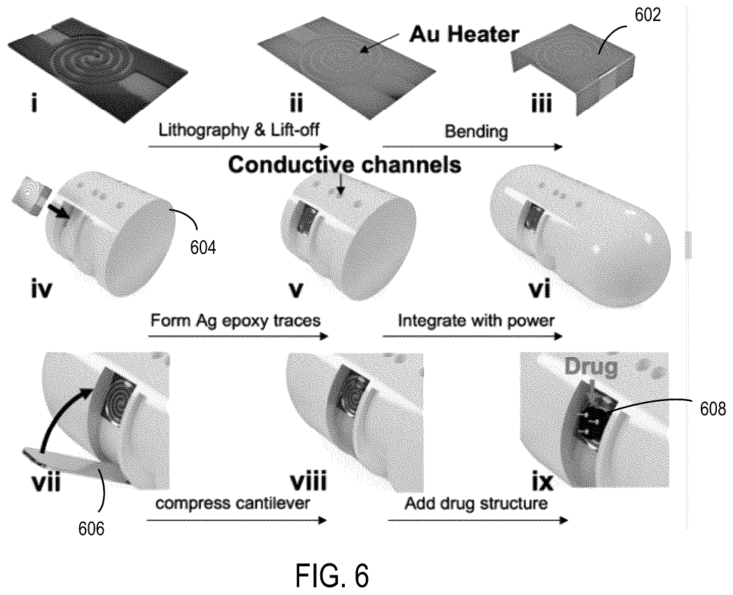

FIG. 6 illustrates details of an example fabrication process of a cantilever actuator. In some examples, this process may begin with using lithography and Au deposition to pattern a resistive heating element 602. Next, the resistive heating element 602 may be placed into an integration module 604. In some examples, the integration module 604 may be manufactured using a 3D printing process. The integration module 604 can use channels filed with Ag epoxy to connect the heater to a power source. Next, the cantilever 606 is inserted into the integration module 604. The cantilever 606 may be compressed and held in place using a low-MP polymer (PCL). Finally, a microneedle-based drug deliver structure 608 is attached to the integration module 604 for prolonged localized drug delivery.

The following paragraphs provide results of the cantilever system and are not intended to be limiting.

### Experimental Procedures

Resistive Heater Fabrication and Evaluation: Resistive heating elements are used to melt the EVA polymer adhesive holding the cantilever in place. The heater demonstrated here has geometry with varied scale and deposition thickness to achieve different thermal dissipation density and resistance, respectively. Heaters were fabricated by lithography and electron beam evaporation deposition of 20 nm Cr followed by Au of varied thickness between 30 nm and 150 nm. Lift-off of the resist was performed by 30 minutes of sonication in acetone. The outer diameter of the coil was varied between 500 μm and 3 mm to optimize heating of the EVA adhesive and reliability. MathWorks MATLAB (Natick, MA, USA) was used to evaluate the dissipation power for heaters of different size and thickness regulated to 3V. Trace resistance was determined using trace geometry, the resistivity of Au (ρ_{Au}=2.44×10⁻⁸ Ω·m), and a previously determined correction factor for a Au heating element power dissipation was calculated via Ohm's Law. Resistance of fabricated heaters was measured (n=4) and compared to understand differences in fabrication repeatability between sizes. Melt time of the 2.5 mm heater (n=3) was assessed with varied resistances and current constraints as represented in FIG. 4F.

Package Fabrication and Assembly: The capsule system is composed of an Energizer 2L76 battery (St. Louis, MO, USA) in series with a magnetic reed switch and the thin-film resistive heating element, packaged in a 3D printed housing with attached drug delivery cantilevers. The module housing the heater and cantilevers was fabricated using a Phrozen Sonic Mini 8K (Hsinchu City, Taiwan) liquid crystal display (LCD) vat photo-polymerization (VPP) 3D printer with FormLabs Surgical Guide v2 resin (Somerville, MA, USA). The module has a 13 mm outer diameter and contains four opposingly oriented recesses to hold the cantilevers below the surface of the capsule before deployment. The recesses have a maximum depth of 3 mm and a width of 3.5 mm, with an overhang on the fixed end of the cantilever to affix the cantilever when tensioned. The module also has ∅=1.5 mm channels leading from heater to the battery cavity, which are filled with MG Chemicals 8330S conductive epoxy (Burlington, ON, CA) to form the electrical interconnects between the heater and battery. Before curing of the Ag-filled epoxy, heaters were placed in the module and 36 AWG insulated wires were inserted into the battery end of the channels. The Littlefuse 6-10 AT normally open reed switch (Chicago, IL, USA) and battery were fused by spot welding using a Ni tab, then soldered to the heater wires. The capsule shell and end cap were fabricated via LCD VPP with a 700 μm wall thickness. The battery and wires were placed in the shell, and the shell and end cap were affixed to the cantilever module with Loctite® UK M-11FL™ medical device urethane adhesive (ISO 10993-5 Biocompatibility; Düsseldorf, DE).

Microneedle Molding: Microneedles were molded in a 11×11 microneedle mold produced by Blueacre Technology (Dundalk, Co. Louth, IE) as described previously by Levy et al.45 The mold contains 121 microneedles spaced by 600 μm on center, with a 300 μm base diameter and a 600 μm height. A 20% w/v solution of polyvinyl alcohol (PVA; Mw 31-50 kDa) from Sigma Aldrich (St. Louis, MO, USA) with contained FD&C Blue #1 dye was poured over the mold then exposed to near vacuum (˜0.03 Atm) to evacuate air from the needle mold. The array was then brought to atmospheric pressure and allowed to dry for 24 hours to remove the water and cast the PVA into the mold. Microneedle arrays were removed from the mold and segmented into separate 3×3 arrays for demonstration.

Cantilever Actuator Assembly: Cantilevers were made from commercially available polymeric films purchased from McMaster-Carr Supply Company (Elmhurst, IL, USA). Polyether ether ketone (PEEK), cellulose acetate (AC), polyester (PES), polycarbonate (PC), and ultra-high molecular weight polyethylene (UHMWPE) were chosen for their biocompatibility and evaluated for mechanical characteristics. The cantilevers were 3×8.5 mm and have 1 mm filleted corners to reduce the chance of bowel damage after deployment. The shape of the cantilever was laser engraved on the film surface using the Glowforge Pro laser cutter (Seattle, WA, USA) then cut to size. The cantilevers were then tucked into the slot on the fixed end of the recess in the cantilever module. 500 μg of ethylene vinyl acetate (EVA) adhesive (The Gorilla Glue Company, Sharonville, OH, USA) was melted on the heater by flowing current, then the cantilever was flexed and attached to the heater while the EVA was in a melt state. Following solidification, the cantilever remained affixed to the heater until deployment via re-melting. After affixing the cantilever, a 3×3 microneedle array was attached to the cantilever using ˜1.5 μg of the water-soluble polymer polyethylene glycol (PEG; Sigma Aldrich, St. Louis, MO, USA) in melt form.

Reed Switch Evaluation: Reed switches of different switching field strength were purchased from DigiKey Corporation (Thief River Falls, MN, USA) and tested to determine the dependence of required field strength for switching and closed resistance on the designed switching strength. Littlefuse Inc. (Chicago, IL, USA) switches designed to close at 6-10 AT, 10-15 AT, and 15-20 AT were evaluated using a permanent NdFeB magnet at variable distance and magnetometer readout. Reed switches (n=3) were placed adjacent to the magnetometer and connected to a multimeter. The magnet was moved closer to the switch until electrical continuity was registered, and the field strength value was recorded. Once open the switch resistance was measured (n=3) to evaluate the impact of switch resistance on current flow in the packaged capsule.

Mechanical and Relaxation Characterization: To determine the deployment force, dynamics, and proneness to stress relaxation predicting long-term reliability, the cantilevers were evaluated using and Instron 5942 ultimate testing machine (UTM; Norwood, MA, USA) with a 5N load cell and a custom 3D printed mandrel with a 5 mm radius of curvature. Based on bulk mechanical properties, the following materials and thicknesses were evaluated (n=3 for all): PEEK (76 μm, 127 μm, 254 μm, 381 μm, 508 μm), UHMWPE (254 μm, 508 μm), HDPE (406 μm, 584 μm), PP (406 μm, 508 μm), AC (127 μm), PES (127 μm), and PC (127 μm). Cantilevers were deployed into the UTM load cell held 1 mm from the surface of the capsule. The force was measured and plotted for a variety of material combinations to reveal the profile and magnitude of deployment force.

Using a custom bending apparatus, the long-term reliability of the most promising cantilever materials and thicknesses were evaluated. The test cantilevers were held at 130° bend angle for 72 hours then removed from the mandrel fixture and imaged to assess the residual bending of each material and thickness variant to indicate the level of stress relaxation and its hindrance to long-term reliability.

Ex Vivo Microneedle Deployment: The capsule was first deployed while submerged in 1□ PBS solution to evaluate watertightness and the ability to deploy while submerged. The actuator was then deployed on a static porcine ex vivo small intestinal tissue (Animal Biotech Industries, Doylestown, PA, USA) to evaluate deployment timing and microneedle penetration in tissue. The deployment and insertion of microneedles into tissue was evaluated using a Chronos 2.1HD high speed camera (Kron Technologies, Burnaby, BC, CA). Following static deployment, the capsule was deployed on an ex vivo tissue simulator to validate the performance in a simulated environment. Translation of 1.4 cm/min was used to simulate the average transit speed in the small intestine, and a force of 500 mN exerted by 51 g of mass approximated the force expected in the small intestine.50 During translation of the assembled capsule, the permanent NdFeB magnet was moved close to the actuator capsule and deployment was monitored following a 10 s hold in proximity of the capsule. Microneedle drug release in the tissue was tracked over a 2-hour period.

### Bi-Layer Ingestible Capsule

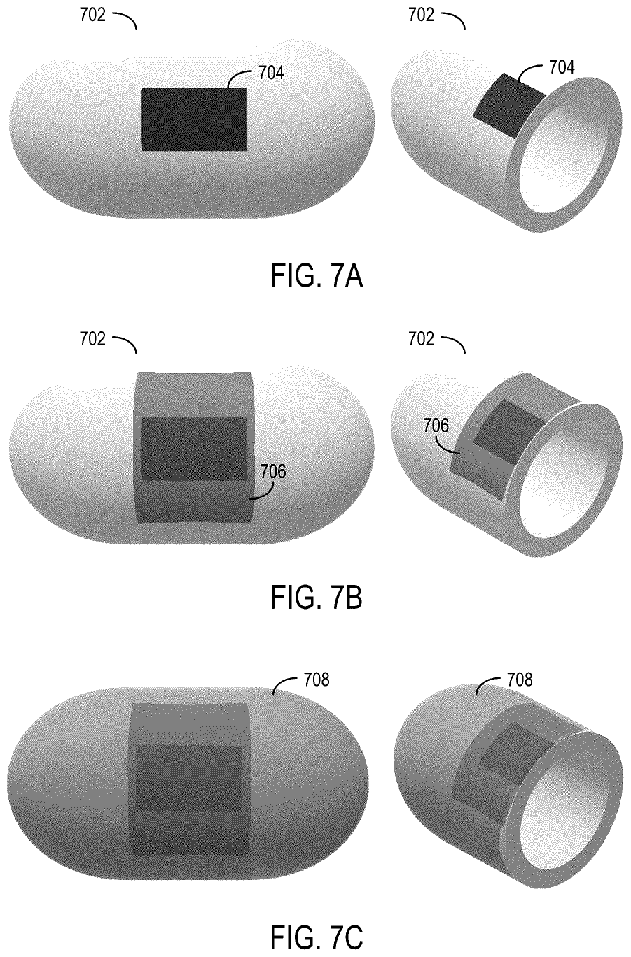

Referring to FIGS. 7A-7C, front and isometric views of the capsule 702 at different stages of FRRB formation are shown. In a non-limiting example, the capsule 702 is configured to be an ingestible capsule. The capsule may be made of polyethylene terephthalate glycol (PETG) or other suitable material for traversing the GI tract. For example, the capsule 702 may be of a biodegradable material to break down and avoid obstructing the GI tract. In another example, the capsule 702 may comprise two or more complementary pieces that are configured to reversibly connect to form the capsule 702.

Further, the capsule 702 comprises one or more features, such as structural features, 704. In a non-limiting example, the one or more features may be one or more active structural features, such as openings. The openings may be any size and shape depending on the desired application as will be described in further detail. In a non-limiting example, an opening may expose a sensor (e.g., impedance sensor, chemical sensor), therapeutic, sampling device (e.g., forceps, pincers, swabs), actuator (e.g., anchor, spring), or any combination thereof. An “active” structural feature thus enables interaction between an electrical or mechanical payload of an ingestible (e.g., capsule). Alternatively, the one or more features 704 may include one or more seams formed between two or more complementary pieces that form the capsule 702 when combined.

As shown in FIG. 7B, a first water-soluble layer 706 at least partially surrounds the capsule 702, wherein the first water soluble layer 706 covers the one or more features 704. Although FIGS. 7B-7C show the first water-soluble layer 706 only covering a small area of the capsule to cover the opening 704, the first water-soluble layer may cover up to the entire surface of the capsule 702.

In a non-limiting example, the first water-soluble layer 706 is a PEG layer (average molecular mass: 4000). The PEG layer 706 provides a rigid support that is capable of covering complex and mm-scale features 704 and allows for their selective exposure in neutral pH environments.

Referring to FIG. 7C, a second pH-responsive layer 708 surrounds the capsule 702 and first-water soluble layer 706. In a non-limiting example, the second pH-responsive layer 708 further comprises one or more coatings applied via dip coating, spray coating, or pan coating. The number of coatings depends on the desired dissolution rate to expose the underlying first water-soluble layer 706 and feature 704. As will be described in further detail, the number of coatings of the second pH-responsive layer may include 1 to 10 coats.

In a non-limiting example, the coatings of the second pH responsive layer 708 may be composed of anionic methacrylic acid copolymers. For example, to target the dissolution of the second pH-responsive layer 708 in the small intestine, an ethyl acrylate and methyl methacrylate copolymer and methacrylic acid-ethyl acrylate copolymer (Eudragit® FL30 D 55) in a 1:1 ratio may be used, which is soluble at pH 5.5 and above. Thus, as the capsule traverses the stomach where gastric fluid lowers the pH to 1.5-3, this second pH responsive layer remains intact and only begins to dissolve as the pH rises as it travels through the small intestine (pH 6-7.4).

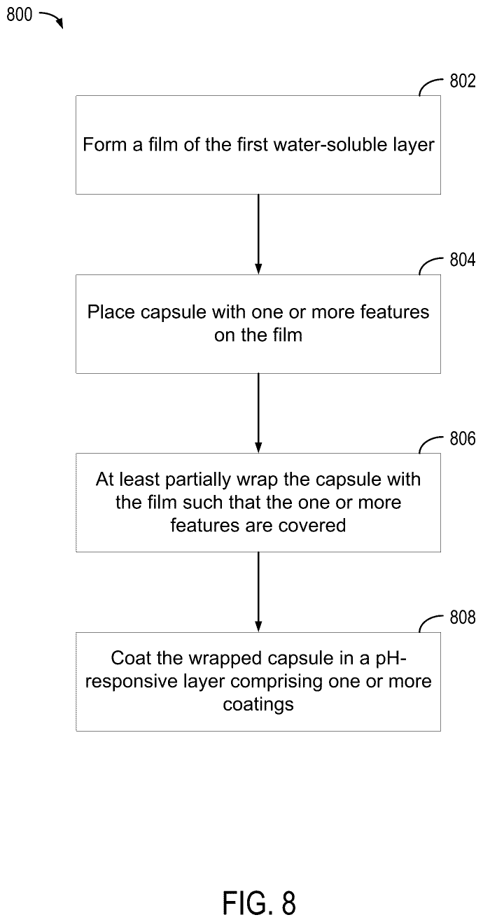

Referring now to FIG. 8, a method 800 of making an ingestible capsule is shown. At step 802, a film of the first water-soluble layer is formed. In a non-limiting example PEG crystals are melted on a polyimide sheet. Thereafter, a capsule including one or more structural features is placed on the film at step 804. At step 806, the capsule is at least partially wrapped with the film, such that the film covers the one or more features. In a non-limiting example, the polyimide sheet is used to wrap the film around the capsule. The capsule, film, and sheet are allowed to cool, and the polyimide sheet is then removed. At step 808, the wrapped capsule is then coated in a pH-responsive layer, wherein the pH-responsive layer comprises one or more coatings. In a non-limiting example, the wrapped capsule is dip coated in ethyl acrylate and methyl methacrylate copolymer and methacrylic acid-ethyl acrylate copolymer (1:1 ratio).

Another aspect of embodiments of the present disclosure, is the combination of pH-responsive and water-soluble materials as a multi-layer coating, to achieve mm-scale freestanding structures that are more geometrically versatile than standard coating solutions alone and less demanding than mechanical systems. Thus, the FRRB gives way to the potential of passively activated functional packaging components.

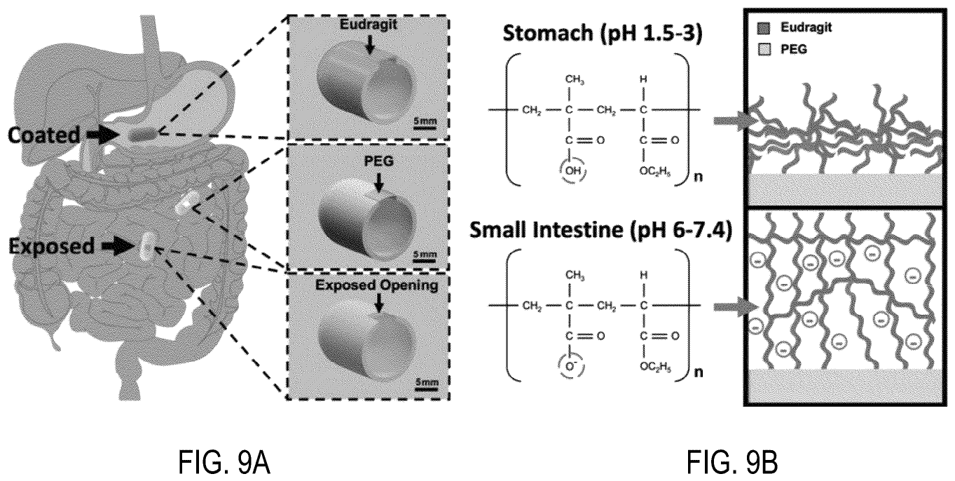

In a non-limiting example, the FRRB comprises a water-soluble rigid PEG layer under a pH responsive Eudragit® FL30 D 55 layer and operates sequentially (FIG. 9A). While in the stomach at pH 1.5-3, the Eudragit® copolymer layer is protonated and compact to shield the underlying PEG layer and capsule from the surrounding environment. Once the capsule reaches the target environment, the small intestine (pH 6-7.4), the copolymer becomes deprotonated and swells to form an open structure. The Eudragit® layer is then able to be removed via a combination of dissolution and abrasion from the intestinal wall. The PEG layer is then dissolved by the aqueous media present in the small intestine.

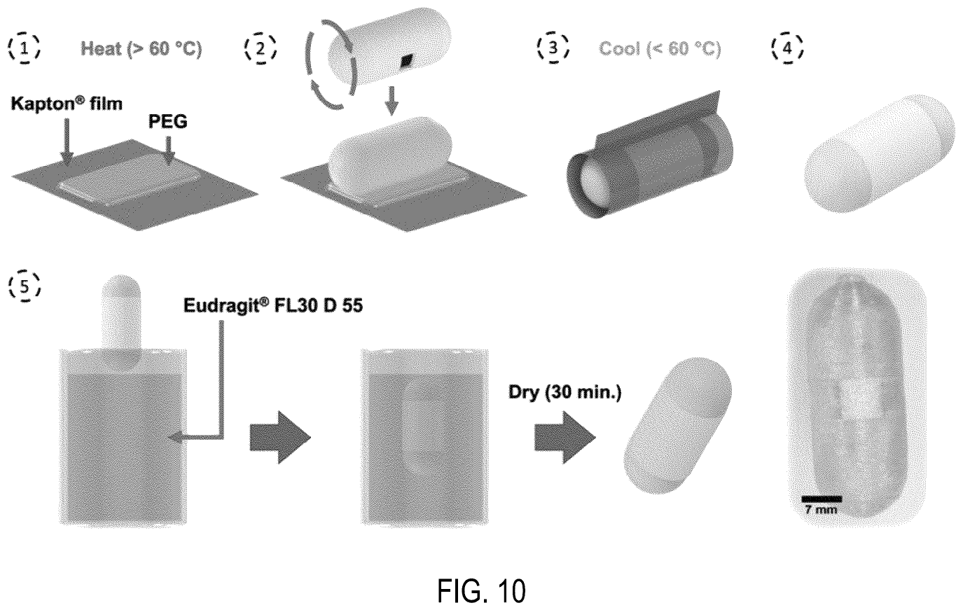

In a non-limiting example, the FRRB is fabricated starting with film transfer of PEG followed by dip coating in Eudragit® FL30 D 55 to establish a freestanding film structure that can be applied to seal mm-scale capsule cavities where commercially available enteric coatings alone cannot (FIG. 10). At steps (1)-(2), PEG is melted onto a Kapton® film and applied over the capsule opening. At step (3), the film is then wrapped tightly around the capsule to significantly reduce heterogeneity in the film formation. Poor adhesion of solid PEG to the transfer film ensures its facile removal without disturbing the PEG layer at step (4). This is followed by dip coating in Eudragit® FL30 D 55 liquid coating formulation at step (5). In an experimental set up to evaluate their ability to produce a sufficient coating layer over capsule openings, the FRRB and standard enteric coatings, Eudragit® FL30 D 55 (FL30) and L 100 55, were applied to square capsule openings ranging from 2×2 mm to 6×6 mm in 3D printed PETG capsules. The FL30 was applied in a 30% w/v aqueous suspension, while the L 100 55 dissolved in solvent at high concentrations (30 and 40% w/v). The capsules were dip coated in high viscosity enteric coating solutions, which has shown to be an effective capsule coating method for preparing small capsule openings for sampling in the small intestine. Neither the FL30 liquid dispersion nor the highly viscous 30% and 40% w/v formulations of L 100 55 were able to coat a 2×2 mm square opening as they lacked appropriate viscosity to form a freestanding film before solvent evaporation. The primary structural PEG layer of the FRRB could easily coat the openings up the maximum 6×6 mm square opening via the melted film transfer process. This was followed by dip coating in the pH responsive FL30 dip solution. The plasticized FL30 formulation as the pH responsive coating layer of the FRRB ensures no organic solvents are included in the films and avoids potential cracking known to occur in thin layers of unplasticized formulations.

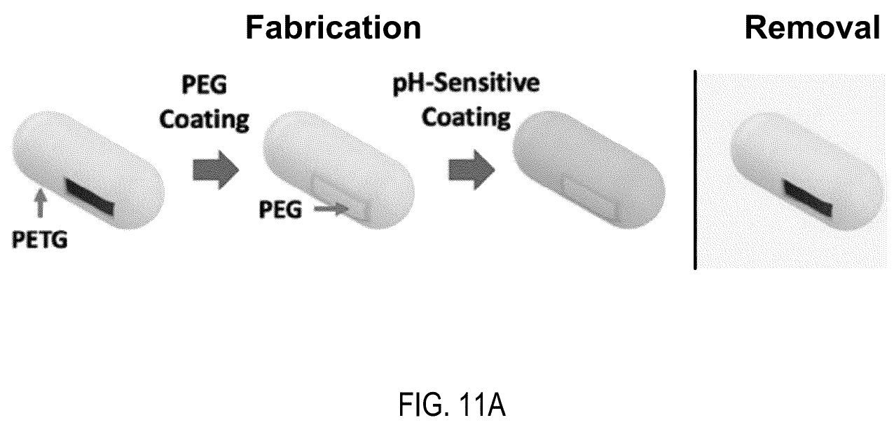

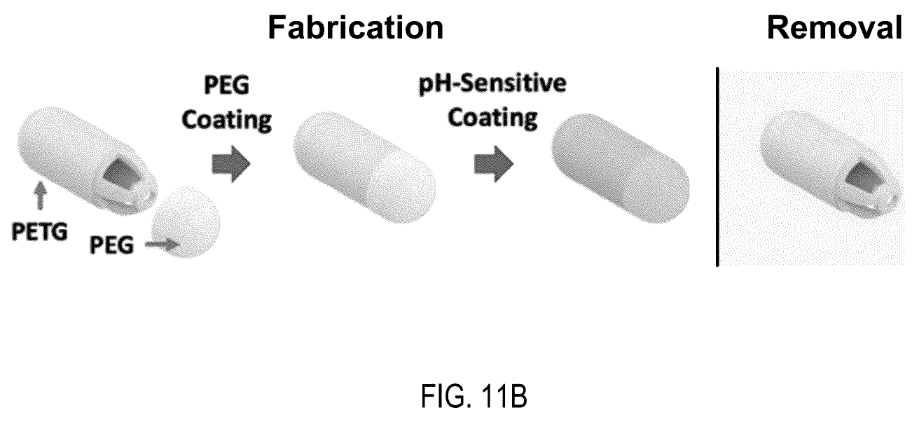

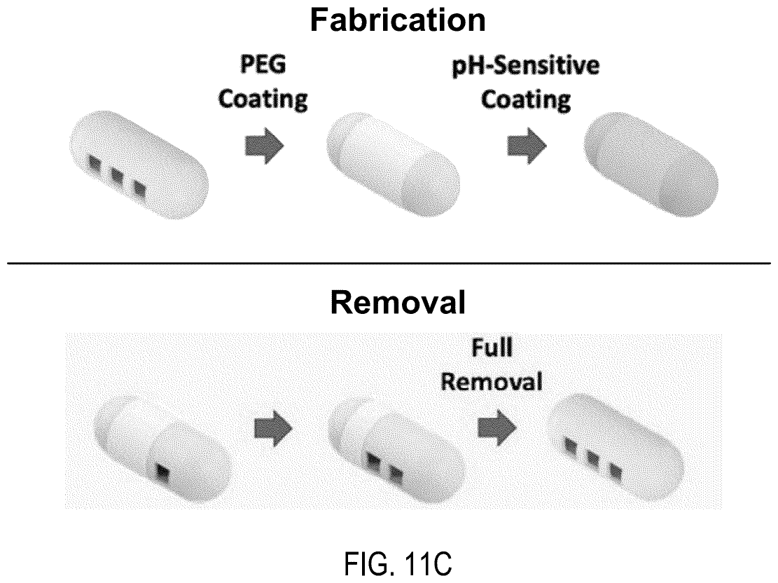

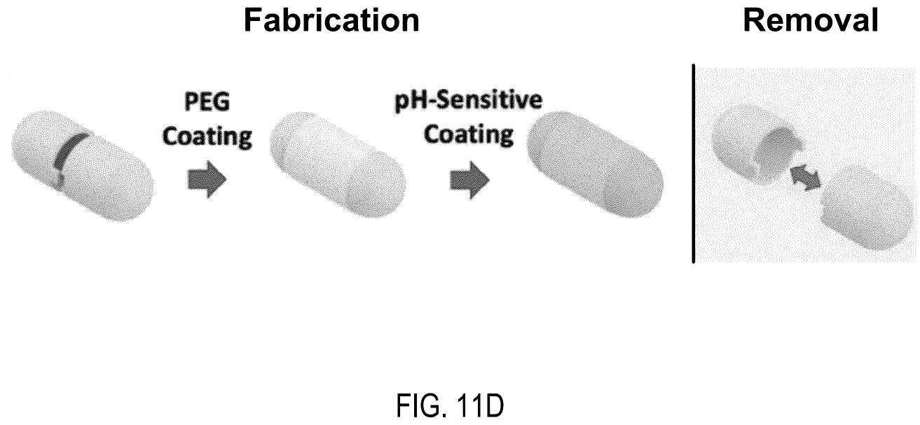

In a non-limiting example, the FRRB can be customized to cover any portion of an ingestible capsule and to form packaging that passively serves a role in capsule operation. This could include operations such as deposition of payloads and exposure of sensors with large surface areas. Coating is facilitated by the forming of the PEG layer into 3D shapes via molding and casting. FIGS. 11A-11D demonstrate the geometric versatility of the FRRB. FIG. 11A shows a slot design. In a non-limiting example, this design may be used with an actuator. In an experimental example, the capsule was 3× dip coated in FL30 as results indicate that three layers would be the minimum to withstand 30 minutes of gastric transit in some embodiments. Such a capsule configuration could be useful in providing selective exposure for actuators that require direct contact with the intestinal wall over large surface areas.

The FRRB can also be applied to capsules designed with functional components that are configured to operate at the ends of the capsule. For example, a dome shaped sheath geometry is illustrated in FIG. 11B. This geometry may be molded from PEG in a PDMS negative mold and fitted over a printed capsule, then dip coated in FL30 (e.g., 3×).

FIG. 11C illustrates a multiport capsule. In a non-limiting example, each opening may be differentially coated utilizing wrapping film. For example, ports may be coated with 1, 2, and 3 layers of FL30 respectively to induce sequential exposure of the three openings. This design could be used to release payloads at different positions in the GI tract with a single capsule.

FIG. 11D illustrates a connector configuration, where two halves of a capsule are connected and held together via the FRRB coating (e.g., 3 FL30 layers). For example, the capsules may be configured such that exposure to the small intestinal pH and abrasive forces of the target environment will cause the halves to separate. The connector design could be used in depositing capsule content for applications like long-term residency.

The following paragraphs provide results of the FRRB capsule and are not intended to be limiting.

### Methods

### Design and Fabrication of 3D Printed PETG Capsule Templates

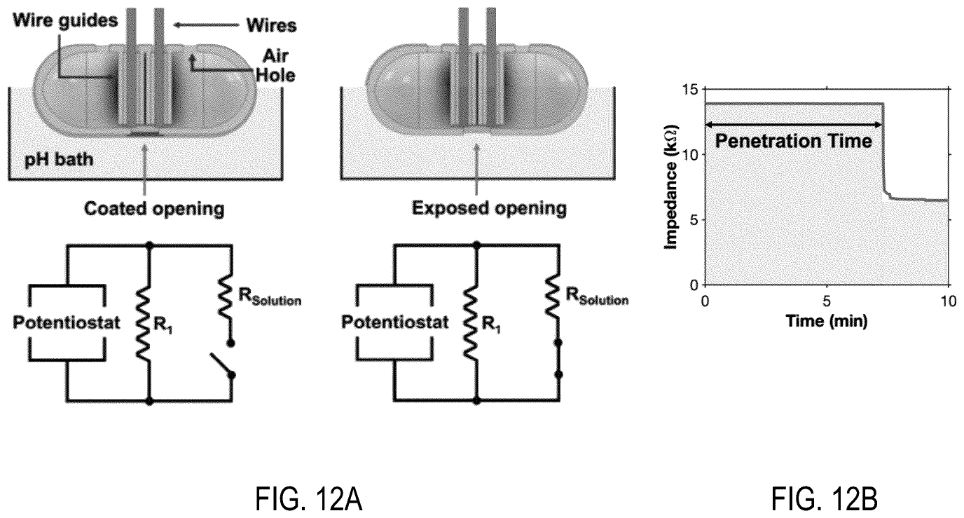

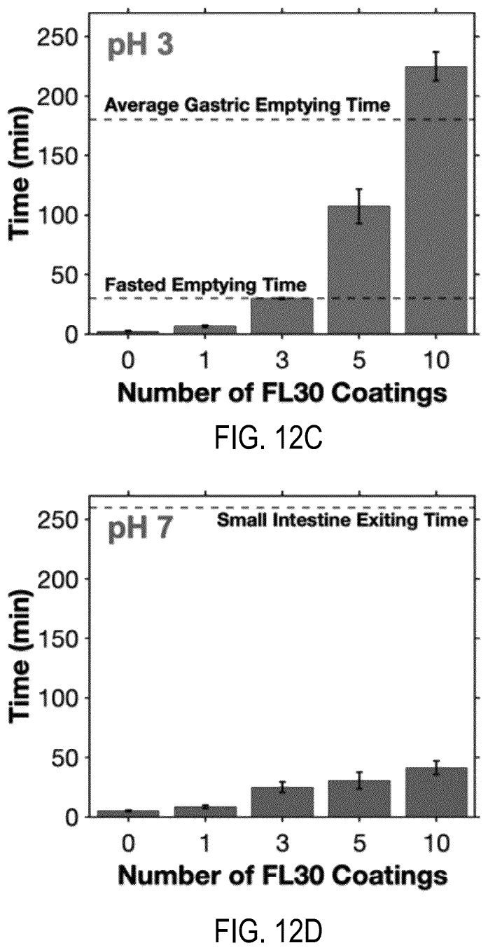

Test capsules (ø=13 mm, L=32 mm) were designed in Autodesk® Inventor™ and 3D printed with fused filament fabrication (FFF) of Polyethylene Terephthalate Glycol (PETG) using a Prusa i3 MK3S+ (Prusa Research, Czechia). A square opening (4 mm×4 mm) was created along the capsule face to evaluate the dissolution of the FBBR. Four additional 2 mm diameter ports were positioned on opposite ends of the test capsule to facilitate the two sensing wires used to indication liquid intrusion, as well as to relieve air pressure and mitigate the formation of an air bubble that might prevent liquid penetration of the capsule opening even after layer removal. The capsules are printed with 1 mm thick internal cylindrical guide structures (inner ø=2.5 mm), to secure the sensing wires near the capsule opening show in FIG. 12A.

### Coating Procedure

FIG. 10 depicts the coating process for each capsule in which, approximately 0.17 g of PEG (Polyethylene glycol 4000, MilliporeSigma, Burlington, MA, USA) was melted on a 1 mil polyimide film, then transferred to the capsule surface to cover the capsule opening. The PEG was allowed to solidify then the polyimide film (Kapton®, DuPont, Wilmington, DE, USA) was removed. Samples were dip coated in Eudragit® FL 30 D-55 (generously donated from Evonik, Essen, Germany), a 30% w/v dispersion of pH-sensitive polymer and plasticizer in water, at a constant entrance and withdrawal speed of 5.5 mm/sec with an immersion time of 1 sec via a custom fabricated dipping apparatus, then allowed to dry at room temperature for 30 min. The dipping process was repeated after drying for multiple coats (3, 5, 10). Wrapping films (Parafilm™, Bemis™, Sheboygan, WI) were used to mask portions of the capsules during the dipping portion of the coating procedure and removed once enteric coating films were completely dry

### Determining pH Responsive Polymer Limits

A 30% w/v standard enteric coating solution was created by dissolving 15 g of Eudragit L 100 55 powder in 50 mL methanol stirred at 300 rpm at a temperature of 60° C. until solute was no longer visible. Capsules were printed with square openings with dimensions ranging from 2×2 mm to 6×6 mm, increasing in increments of 1 mm. These capsules are dip coated with in Eudragit FL30 D-55, and the FRRB then assessed via observation to determine coating success. Capsules were sectioned and inspected to determine whether internal leakage occurred.

### pH Bath Coating Penetration and Removal Test

Coating penetration time is evaluated by incubating coated capsules baths of 0.1 M acetate (pH 3) and Dulbecco's phosphate buffer saline (DPBS, pH 7) at physiological temperature (37° C.). Baths are stirred via a magnetic bar at 250 rpm while temperature is maintained by an ETS Model 5506 Environmental Chamber from Electro-Tech Systems Inc. (Perkasie, PA, USA). Wires were inserted into the capsule through the guides, resting just outside of the opening. These wires are connected in parallel to a 20 k or 100 k resistor which is connected to the VSP-300 potentiostat from Bio-Logic Science Instruments (Seyssinet-Pariset, France) (FIG. 12A). The impedance of the system at frequencies of 10 or 10 k Hz were obtained via and EC-Lab software from Bio-Logic Science Instruments (Seyssinet-Pariset, France) during the bath. Experiments are terminated after a large drop in the impedance is observed, indicating liquid penetration of the capsule and connection of the two wires.

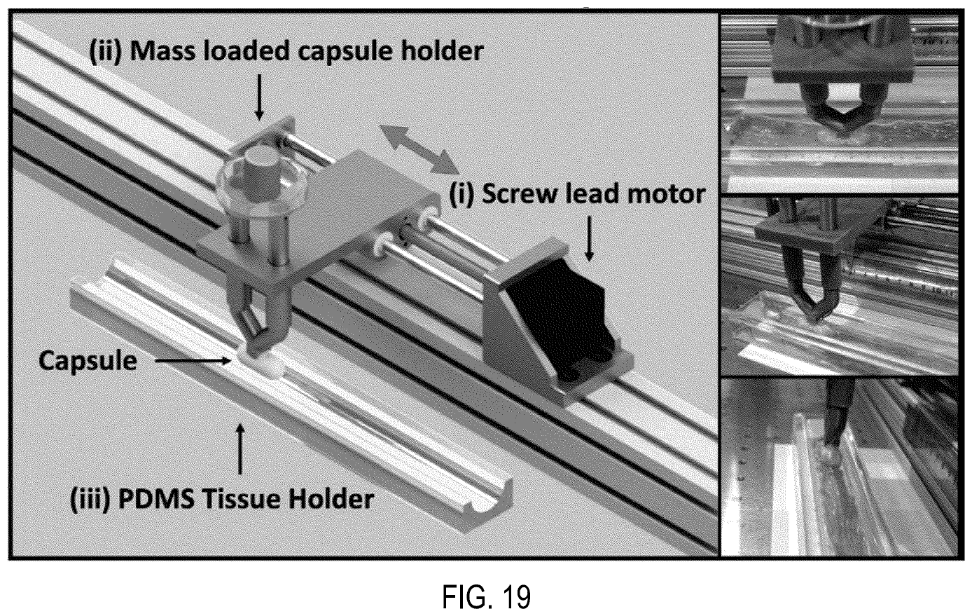

To evaluate the contribution of abrasive interactions between the GI wall and the capsule that facilitates coating removal, a custom fabricated GI translation simulator was utilized which translates the capsule across a silicone small intestine phantom (SurgiReal, Inc., Loveland, CO, USA) (FIG. 19) in 7 mL of DPBS. Capsules are translated at average peristaltic speed of 1.4 cm/min47 with 1.9 N/cm force50 is applied downward via the sample holder.

### Results

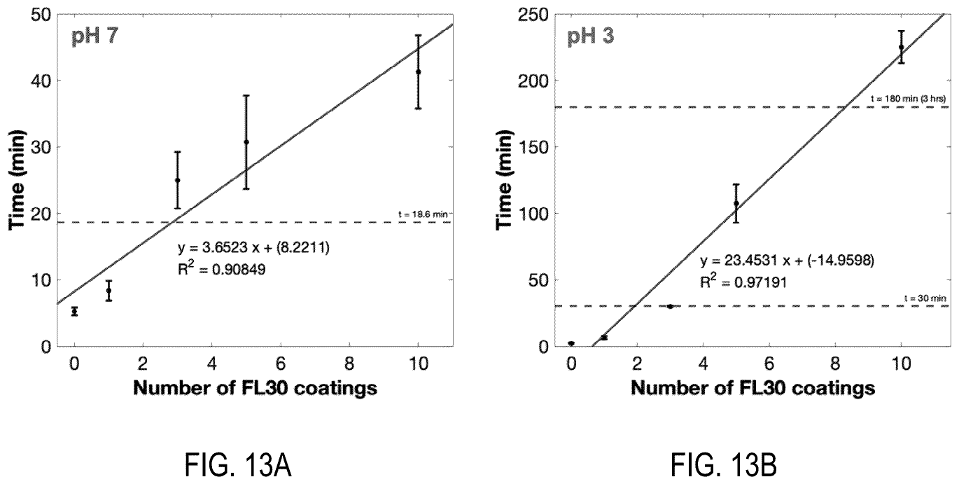

The ability of the FRRB to protect internal capsule components from GI environmental fluid exchange was evaluated by incubating coated capsules in pH baths simulating GI transit and measuring the time to bilayer penetration. Capsules were evaluated in a simulated stomach bath at pH 3 and a simulated intestine bath at pH 7. Wires were inserted into test capsules and positioned against the internal surface of the capsule adjacent to the capsule opening (FIG. 12A). These wires were connected in parallel to a potentiostat which measured circuit impedance throughout the incubation period. The penetration time of the FRRB is determined as the time at which a sharp decline in impedance is observed, resulting from the electrical connection across the wires formed by the pH bath fluid (FIG. 12B). Capsules were dip coated with multiple layers of Eudragit® FL30 (0, 1, 3, 5, and 10) to modulate the penetration time as previously shown with similar enteric coatings. GI transport can differ based on the state of the system prior to ingestion. Ingestible capsules are generally administered to patients in a fasted state, where there is no food present prior to administration. Capsules dip coated in FL30 at least three times met a sufficient protection time, 29.9±1.3 min, in simulated gastric fluid to allow for transport to the small intestine within a fasted system (FIG. 12C). For normal fed systems, gastric transport time is about 2 hours. To achieve sufficient protection for the average fed system the capsule would have to be coated near ten times, where the penetration time was shown to be 225±24 min. In the simulated intestinal environment, the FRRB was penetrated long before the estimated intestinal emptying time of 260 min, as the ten FL30 layer coating was penetrated at 41.3±11 min (FIG. 12D). The mean FRRB penetration time and standard deviation for all evaluated number of FL30 layers are summarized in FIGS. 13A-13B.

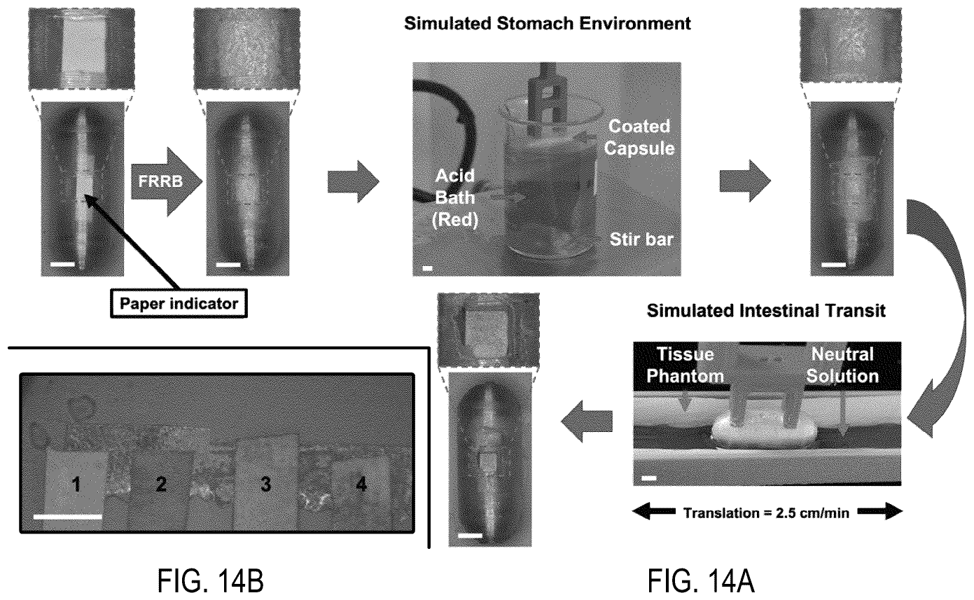

The region-targeted exposure of capsule contents via the FRRB was demonstrated using solution color indicators to reflect whether exposure occurred in the acid bath or neutral intestinal phantom. A paper strip was packaged inside a 5× coated capsule and the capsule was subjected to simulated transit through the GIT using reported ingestible capsule transit conditions. First, the capsule was incubated for 30 minutes in a stirred acidic bath, simulating gastric passage in a fasted system (FIG. 14A). The capsule was then translated across simulated GI tissue in neutral solution to simulate intestinal transit. Acidic solution was dyed red and neutral solution was dyed blue to indicate region of exposure. Visual inspection of the capsule showed that the bilayer remained intact and unpenetrated after acidic incubation, however, was exposed after 5 min of transit as was indicated by the paper strip being stained blue after translation (FIG. 14B).

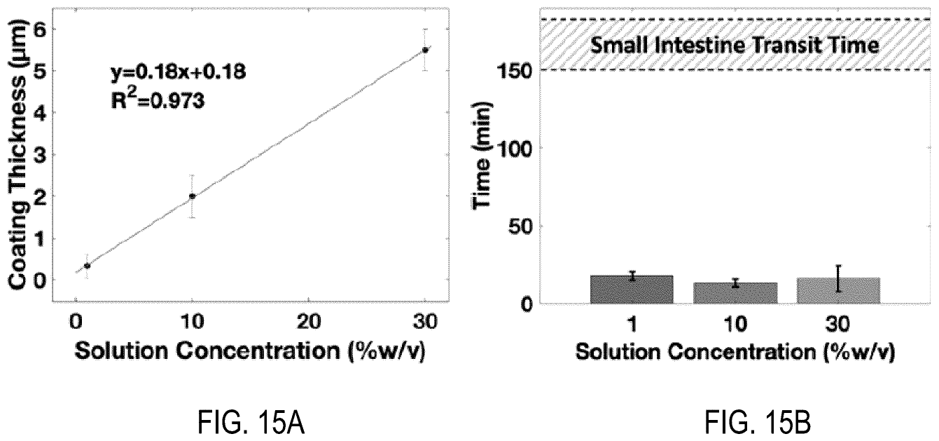

Dip-coated film thickness was found to be 0.3±0.2 μm, 2.0±0.3 μm, and 5.5±0.3 μm for 1, 10, and 30% w/v Eudragit® FL 30 D-55 solutions, respectively, showing linear correlation (R²=0.973) with a proportionality constant of 0.18 μm/(% w/v) (FIGS. 15A-15B). Acid bath Dissolution experiments showed that capsules coated with the PEG Eudragit® FL 30 D-55 bilayer were not permeable after 3 hours, while capsules coated in PEG alone were permeated within 3 min.

Mechanical removal of FRRB coatings with 1%, 10%, and 30% Eudragit® layers showed mean removal times of 17.7±1.8 min, 13.1±1.3 min, and 16.0═4.9 min, respectively (FIG. 15B), with an average of 15.6 min. This is approximately 1/10th of the range of small intestine transit times previously demonstrated for a capsule, labeled as a gray band. Thus, this system can protect capsule systems (actuators, sensors, openings) in transit through the low-pH stomach region and then rapidly reveal active subcomponents shortly after entering the small intestine target region.

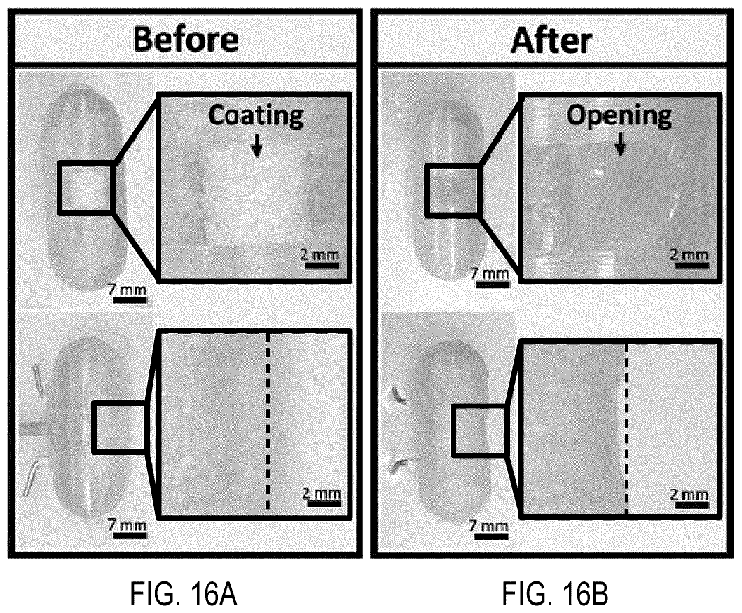

FIG. 15B showed that Eudragit® coating thickness has negligible effect on the removal time of the bilayer, suggesting that the thicker PEG support layer is mostly responsible for removal time, and the Eudragit® is only a protective coating to inhibit dissolution of the PEG layer. The Eudragit® material showed rapid swelling and softening once placed on the neutral intestinal tissue, followed by rapid removal after the start of translational motion. The underlying PEG layer remained robust for ˜15 min after Eudragit® removal, followed by PEG disintegration, as can be seen in the image of a capsule sample after translation across ex-vivo tissue in FIGS. 16A-16B.

FIGS. 17A-17B show validations of the ability of the capsule configurations to expose the capsule cavity in the target environment, dome, connector, and multiport capsules were evaluated in the GI translational simulator translated across a small intestinal tissue phantom to assess coating removal time. Each of the functional packaging configurations resulted in FRRB removal in under 45 minutes, suggesting targeted exposure within transit through the small intestine (FIGS. 17A-17B). Openings on the multiport capsule were exposed in order from fewest number of FL30 coatings to greatest number of FL30 coatings, as expected.

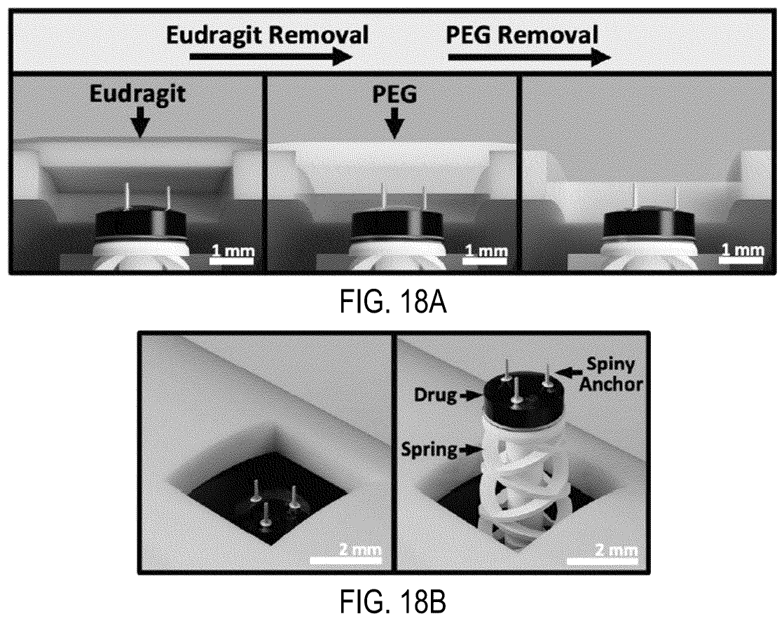

FIGS. 18A-18B illustrate a non-limiting example of an FRRB capsule whereby a spring within the capsule acts as an actuating drug or payload delivery device upon exposure of the opening in the capsule.

FIG. 19 is a diagram and images of a GI simulator system used to evaluate and demonstrate the characteristics of the FRRB capsule.

### Discussion of Certain Examples and Experiments

The devices and methods described here provide a versatile and accessible bilayer packaging technology for the protection and targeted exposure of ingestible capsule functional components. The strategy requires no active stimulation and is readily accessible via facile fabrication techniques like dip coating, molding, and film transfer. Neither of the materials that comprise the FRRB have been reported to have significant electromagnetic shielding properties and thus permits wireless communication for potential real-time in situ sensor feedback or data transmission. The vast molding possibilities for the rigid PEG layer allows the FRRB to form a myriad of shapes that can be used to create functional packaging components of an ingestible capsule. This was demonstrated by fabricating and evaluating four different and non-limiting capsule designs coated with the FRRB. All four designs, on average, were found to expose the internal capsule components in under 40 minutes of transit at neutral pH, implying the potential for release long before the small intestine exiting time (260 min). This is comparable with total dissolution time of other non-freestanding pH-responsive films used for small intestinal targeting. The performance of the FRRB outside of normal physiological conditions has yet to be explored, however, it is expected that a reduction in physiological pH or contact force would result in prolonged removal. Reduction of pH would decrease pH responsive film expansion while a reduction in contact force would decrease shearing. In the inverse case, an expedited removal is expected. The characterization of the FRRB confirms the trend that increasing the number of film coatings increases the time to internal leakage. Thus, the FRRB can be tuned to target specific subregions of the intestines via modulating the dip coating procedure or utilizing an alternate pH responsive polymer formulation such as Eudragit S 100 for large intestinal targeting.

The performance of the FRRB in the GI environment is determined by the dynamic array of conditions such as temperature, pH, and mechanical abrasions a capsule will be subject to that contribute to film leakage and removal. The device and methods described herein are designed to recapitulate GI conditions of a fasted state, the state in which endoscopic capsules are administered, on the benchtop. The journey through the GI tract from entry of the esophagus to passing out of the large intestine sees changes in pH and mechanical perturbations, however, body temperature is well maintained at 37° C. throughout. On average, transit through the esophagus is less than a minute at a neutral pH, which would have negligible effects on the FRRB given the results of the bilayer penetration and translation experiments. The stomach environment poses little challenge to the removal of the FRRB given its acidic pH and minimal interfacial abrasion due to the geometry and large volume relative to standard ingestible capsules. This is reflected in the results of the bilayer penetration experiments as well as in the literature using related pH-responsive films utilizing similar experimental means of validation. FRRB removal begins in the small intestinal environment, where it is exposed to a neutral pH for the duration of transit and high contact forces (0.9-2.9 N/cm). By applying the mean contact force (1.9 N/cm) and translating at the mean translation speed expected in the small intestine (1.4 cm/min) in a neutral pH environment, a more complete simulation of relevant factors for removal is achieved when compared to stirred solutions alone.

At lower pH-responsive coating layer thickness (0-3), FRRB penetration at both acidic and neutral pH are relatively similar, although FL30 films were still unremoved after penetration in the acid bath. This may imply that the mode of transport across the coating at these ranges are more dominated by FL30 film porosity rather than dissolution. Penetration time quickly diverges in the 5-10 coating layer range. Bilayer penetration time may be increased by decreasing dip speed. The dip speed used to prepare all samples (5.5 mm/sec) would produce films under the viscous drag regime of dip coating, leading to impregnated pores in the film. These pores could lead to premature exposure of the PEG layer, ultimately accelerating removal time. Decreasing the dip speed below 1 mm/sec would produce films within the intermediate or capillary regime of dip coating, reducing the presence of the impregnating pore structures.

Experimental results indicated that FRRB penetration time did not always coincide with full exposure of the capsule cavity as partial gaps in the FL30 layer provided access for bath solution to dissolve the underlying PEG. This may be due to excess plasticizer in the FL30 film delaying dissolution. However, removal in the small intestine is not only facilitated by the dissolution of the pH-responsive polymer but also the abrasive interfacial forces present between the film and GI lumen. Translation on the GI simulator better approximates the removal in the target region as the polymer films expand and become more susceptible to mechanical removal in addition to dissolution. With these mechanisms of removal in mind, the FRRB design may be improved to release earlier in small intestinal transit by adding additional materials to improve adhesion to GI walls such as mucoadhesives. Increased adhesion would increase the effect of frictional force on the pH responsive layer, facilitating earlier removal. Chitosan is a mucoadhesive biopolymer that has been used in pH-responsive drug delivery systems and shown not to affect the pH-responsiveness of underlying layers.

As described herein, the FRRB can protect large areas of a capsule establishing a gap between itself and internal capsule components. The ability to protect large surface areas without direct contact with the underlying components could provide a simple packaging solution for sensors that have been functionalized with fragile surface modifications such as antibody or aptamer-based sensors. These sensors can be subject to damage or removal via exposure to harsh environments or blocked from active binding to target molecules due to potential residue from unremoved plasticizers typically used with enteric coatings to modulate mechanical flexibility. Additionally, the FRRB can be used in concert with triggered site-specific drug delivery mechanisms as was demonstrated with thermomechanical actuator. Other methods that utilize enteric coatings for drug release rely on passive diffusion through the coating as it dissolves which has been shown to disperse drug over a period of around 20 min. The modulation of release times by differentially coating various capsule openings can be tailored toward the development of controlled multi-release drug delivery capsules and resident devices. Overall, the FRRB provides a versatile packaging solution that can be readily applied to any ingestible capsule platform.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

1. A device comprising:
a capsule configured to be ingested by a patient and traverse the patient's gastrointestinal tract, the capsule comprising:
at least two layers forming an outer surface of the capsule:
a first water-soluble layer at least partially surrounding the capsule, wherein the first water-soluble layer covers one or more features of the capsule; and
a second pH-responsive layer surrounding the capsule and first water-soluble layer; and
a payload contained by at least a portion of the outer surface of the capsule, the payload comprising at least one electrical or mechanical element.

2. The device of claim 1, wherein the first water-soluble layer includes polyethylene glycol (PEG).

3. The device of claim 1, wherein the second pH-responsive layer further comprises one or more coatings.

4. The device of claim 3, wherein the one or more coatings include anionic methacrylic acid copolymers.

5. The device of claim 4, wherein the methacrylic acid copolymers include ethyl acrylate and methyl methacrylate copolymer, methacrylic acid-ethyl acrylate copolymer (1:1).

6. The device of claim 4, wherein the pH-responsive layer is soluble at pH 5.5 and above.

7. The device of claim 1, wherein the capsule comprises a housing onto which the outer surface is disposed, the housing having one or more openings to be exposed upon dissolution of the at least two layers of the outer surface of the capsule.

8. The device of claim 7, wherein the at least one electrical or mechanical element comprises a sensor, a therapeutic, a sampling device, an actuator, or a combination thereof exposed by the one or more openings.

9. The device of claim 8, wherein the actuator includes a spring configured to extend through the opening.

10. The device of claim 1, wherein the capsule further comprises two or more complementary pieces configured to reversibly connect to form the capsule.

11. The device of claim 10, wherein, when connected, the two or more complementary pieces form one or more seams between each of the two or more pieces to form a housing of the capsule.

12. A method of making an ingestible capsule, the method comprising:
forming a film of a first layer, the film being water-soluble;
placing a capsule including one or more active structural features on the film;
at least partially wrapping the capsule with the film such that the film covers the one or more features; and
coating the wrapped capsule in a second, pH-sensitive layer, the layer comprising one or more coatings.

13. The method of claim 12, wherein coating the wrapped capsule includes dip coating, spray coating, or pan coating.

14. The method of claim 12, wherein the first water-soluble layer includes polyethylene glycol (PEG).

15. The method of claim 12, wherein the one or more coatings include anionic methacrylic acid copolymers.

16. The method of claim 15, wherein the methacrylic acid copolymers include ethyl acrylate and methyl methacrylate copolymer, methacrylic acid-ethyl acrylate copolymer (1:1).

17. The method of claim 15, wherein the second, pH-sensitive layer is soluble in at and above pH 5.5.

18. The method of claim 12, wherein the one or more active structural features includes one or more openings.

19. The method of claim 18, wherein the capsule further comprises a sensor, a therapeutic, a sampling device, an actuator, or a combination thereof positioned to be exposed by the one or more openings upon dissolution of the first layer and the second layer.

20. The method of claim 19, wherein the actuator includes a spring configured to extend through the opening.

21. The method of claim 12, wherein the first layer and second layer have thicknesses such that the layers will not fully dissolve in a stomach, but will be fully dissolved and expose the active structural feature exposed to the interior of an intestinal lumen after the stomach.

22. The method of claim 21, wherein the one or more features include one or more seams between each of the two or more pieces forming the capsule.

23. A device comprising:
a capsule configured to be ingested by a patient and traverse a patient's gastrointestinal tract, the capsule comprising:
a battery;
a switch electrically connected in series with the battery;
a resistive microheater electrically connected in series with the switch;
an actuatable arm having a first end and a second end, the first end of the arm affixed to the capsule; and
an array of microneedles coupled to the second end of the arm and configured to penetrate intestinal tissue of the patient's gastrointestinal tract upon actuation of the arm;
wherein the second end of the actuatable arm is releasably coupled to the resistive microheater via an adhesive for actuation.

24. The device of claim 23, wherein the array of microneedles is a 2×2 mm patch.

25. The device of claim 23, wherein the resistive microheater is formed from a conductive material in a patterned trace.

26. The device of claim 25, wherein the trace is a spiral having has an outer diameter between 1 mm and 2.5 mm, and wherein the resistive microheater comprises a metal trace with a thickness of less than 80 nm.

27. The device of claim 23, wherein the adhesive is ethylene vinyl acetate (EVA).

28. The device of claim 23, wherein the resistive microheater is configured to melt the adhesive when the switch is closed.

29. The device of claim 28, wherein the arm comprises a main portion that is shaped such that, prior to melting of the adhesive, the arm is under mechanical tension and after melting of the adhesive, will release to an extended position distal from the capsule.

30. The device of claim 23, wherein the capsule is made from polyethylene terephthalate glycol (PETG).

31. The device of claim 23, wherein the capsule further comprises one or more layers made of at least one of: polyether ether ketone (PEEK), cellulose acetate (AC), polyester (PES), polycarbonate (PC), and ultra-high molecular weight polyethylene (UHMWPE).

32. The device of claim 23, wherein the actuatable arm is made from a polymeric film.
